(19) United States
(12) Patent Application Publication
(10) Pub. No.: US 2025/0002829 A1
(43) Pub. Date: Jan. 2, 2025

(54) SEAWEED BIOREACTOR APPARATUS, METHODS, AND SYSTEMS

(71) Applicant: Industrial Plankton Inc., Victoria (CA)

(72) Inventors: Robert Roulston, Victoria (CA); Stuart de Haas, Victoria (CA); Shawn Glover, Pacific Grove, CA (US); William Leite, Brentwood Bay (CA); Cameron Gregory, Victoria (CA); Jennifer Long, Victoria (CA)

(73) Assignee: Industrial Plankton Inc., Victoria, BC (CA)

(21) Appl. No.: 18/577,270

(22) PCT Filed: Jul. 7, 2022
(86) PCT No.: PCT/CA2022/051070

§ 371 Date: Jan. 8, 2024

Related U.S. Application Data
(60) Provisional application No. 63/218,952, filed on Jul. 7, 2021.

(51) Int. Cl. C12M 1/00; C12M 1/26; C12M 1/34; C12M 1/36

(52) U.S. Cl. CPC C12M 21/02; C12M 23/22; C12M 29/14; C12M 31/10; C12M 33/12; C12M 41/10; C12M 41/16; C12M 41/32; C12M 41/34; C12M 41/36; C12M 41/48

(57) ABSTRACT

Aspects of seaweed bioreactor apparatus, methods, and systems are described. One aspect is a method comprising: scaling a seaweed biomass and a culture medium in a light-transmitting vessel located in a light-controlling enclosure; and sampling or harvesting the seaweed biomass without unsealing the light-transmitting vessel by: causing, with a controller, a light source to output an artificial light into the light-controlling enclosure that illuminates the seaweed biomass; outputting, with a sensor in communication with the controller, sensory data associated with the culture medium; modifying, with the controller, at least one of the artificial light or the culture medium responsive to the sensory data; intermittently mixing and cutting, with the controller, the seaweed biomass in the light-transmitting vessel; and removing a portion of the seaweed biomass from the light-transmitting vessel and the light-controlling enclosure. Related seaweed bioreactor apparatus, methods, and systems also are described.

### BACKGROUND

### Field

This disclosure relates generally to aspects of a seaweed bioreactor. Particular aspects relate to seaweed bioreactor apparatus, methods, and systems.

### Description of Related Art

The lifecycle of seaweed is complex, making it difficult to farm on an industrial scale. For example, many seaweed species have a biphasic or haploid-diploid life cycle, in which the species alternates between haploids, or cells having a single set of chromosomes in the nucleus; and diploids, or cells having two sets of chromosomes in the nucleus. Under optimal conditions in terms of light, nutrients, and other factors, diploids may reliably produce haploid spores by meiosis involving divisions of the cell nucleus; the haploid spores may reliably develop into male and female haploid adults called gametophytes; and the gametophytes may reliably produce eggs and sperm that unite to grow into diploid adults called sporophytes.

The optimal conditions for seaweed growth can be difficult or near impossible to maintain in natural environments, such as seaweed farms, where variables such as light and nutrients cannot be carefully controlled, making it difficult to reliably sustain growth during each stage of the seaweed lifecycle. For this reason, many seaweed farms are looking for new ways to produce gametophytes and/or sporophytes, such as in controlled environments that are engineered to establish and maintain the optimal conditions for seaweed growth in a cost-effective manner.

### BRIEF SUMMARY

One aspect of this disclosure is a method. For example, the method may comprise: operatively sealing a seaweed biomass and a culture medium in a light-transmitting vessel located in a light-controlling enclosure; and harvesting the seaweed biomass without opening the light-controlling enclosure or introducing outside air to the light-transmitting vessel by: outputting, with a controller, an artificial light into the light-controlling enclosure to illuminate the seaweed biomass through the light-transmitting vessel; intermittently mixing and cutting, with the controller, the seaweed biomass in the light-transmitting vessel; and removing a portion of the seaweed biomass from the light-transmitting vessel while maintaining a positive pressure in the light-transmitting vessel. Another aspect of this disclosure is another method. For example, the method may comprise: sealing a seaweed biomass and a growth medium inside of a light-transmitting vessel located in a light-controlling enclosure; outputting, with a controller, an artificial light into the light-controlling enclosure to illuminate the seaweed biomass through the light-transmitting vessel; intermittently mixing and cutting, with the controller, the seaweed biomass in the light-transmitting vessel; and removing a portion of the seaweed biomass from the light-transmitting vessel while maintaining a positive pressure in the light-transmitting vessel.

In keeping with these methods, operatively sealing the seaweed biomass may comprise pumping, with the controller, into the light-controlling enclosure, any combination of seaweed diploids, seaweed haploid spores, female seaweed haploid adults, male seaweed haploid adults, seaweed eggs, seaweed sperm, and seaweed sporophytes. The methods may comprise regulating a temperature of the culture medium, such as by regulating the temperature of the culture medium by interacting exterior surfaces of the light-transmitting vessel with a heat transfer fluid and/or circulating the heat transfer fluid through a thermal jacket surrounding the light-transmitting vessel.

The methods may comprise outputting the artificial light from one or more LEDs located in the light-controlling enclosure, such as by outputting different intensities or wavelengths of the artificial light with the one or more LEDs at different times. For example, the methods may comprise switching, with the controller, between the different intensities or wavelengths of the artificial light responsive to a sensor operable to output data associated with one or both of the seaweed biomass and the culture medium. As a further example, the methods may comprise determining, with the controller, based upon data output from a pH sensor located in the light-transmitting vessel, a pH level of the culture medium in the vessel; and switching between the different intensities or wavelengths of the artificial light responsive to the pH level of the culture medium. The methods also may comprise determining, with the controller, based upon data output from an optical sensor, an opacity of the seaweed mass in the light-transmitting vessel; and switching between the different intensities or wavelengths of the artificial light responsive to the opacity of the seaweed mass.

Intermittently mixing and cutting may comprise rotating, with the controller, a mixing edge of a blade in a direction at a first RPM for a first time to mix the seaweed mass in the light-transmitting vessel; and rotating, with the controller, a cutting edge of the blade in a different direction at a second RPM for a second time to cut the seaweed mass into smaller pieces in the light-transmitting vessel. The second RPM greater than the first RPM.

Another aspect of this disclosure is another method. For example, the method may comprise: sealing a seaweed biomass and a culture medium in a light-transmitting vessel located in a light-controlling enclosure; and sampling or harvesting the seaweed biomass without unsealing the light-transmitting vessel by: causing, with a controller, a light source to output an artificial light into the light-controlling enclosure that illuminates the seaweed biomass; outputting, with a sensor in communication with the controller, sensory data associated with the culture medium; modifying, with the controller, at least one of the artificial light or the culture medium responsive to the sensory data; intermittently mixing and cutting, with the controller, the seaweed biomass in the light-transmitting vessel; and removing a portion of the seaweed biomass from the light-transmitting vessel and the light-controlling enclosure.

Sealing the seaweed biomass may comprise adding seaweed cells to the culture medium. Sealing the seaweed biomass comprises adding seaweed cells to the culture medium without opening the light-controlling enclosure or unsealing the light-transmitting vessel. Adding seaweed cells may comprise adding one or more of diploids, seaweed haploid spores, female seaweed haploid adults, male seaweed haploid adults, seaweed eggs, seaweed sperm, seaweed sporophytes, seaweed zoospores, gametophytes, eggs, gametes, or sporophytes. Adding seaweed cells may comprises adding one or both of male gametophytes and female gametophytes.

Sealing the culture medium may comprise maintaining a biosecure environment inside the light-transmitting vessel. Adding the amount of the culture medium may comprise adding an amount of water. Sampling or harvesting the seaweed biomass may comprise removing the portion of the seaweed biomass without introducing unfiltered ambient air to the light-transmitting vessel. The method may comprise only opening the light-controlling enclosure after a period spanning months or years. The method may comprise unsealing the light-transmitting vessel after a period spanning months or years. Causing the light source to output the artificial light may comprise causing a LED to output the artificial light. Causing the LED to output the artificial light may comprise causing a spectrum selectable LED to output the artificial light. The method may comprise causing, with the controllers, the multi-color LED to output a first hue of the artificial light for a first period; and causing, with the controllers, the multi-color LED to output a second hue of the artificial light for a second period. Outputting the second hue of the artificial light for the second period comprises changing the seaweed biomass to sexual.

The light-controlling enclosure may comprise a light-filtering window and the method may comprise admitting a filtered portion of ambient light into the light-controlling enclosure through the light-filtering window. Admitting the filtered portion of ambient light may not comprise changing the seaweed biomass to sexual. Modifying the culture medium may comprise modifying, with the controller, a flow of CO₂ to culture medium. Outputting the sensory data may comprise outputting a pH level of the culture medium. The method may comprise adjusting, with the controller, the at least one of the artificial light or the culture medium responsive to pH level of the culture medium. The method may comprise monitoring, with the controller, the pH level of the culture medium; and modifying, with the controller, the culture medium by modifying a flow of CO₂ to the culture medium responsive to the pH level of the culture medium until it approaches a target pH level of the culture medium. The method may comprise determining, with the controller, a CO₂ demand of the seaweed biomass responsive to the pH level of the culture medium; and modifying, with the controller, the culture medium responsive to the CO₂ demand. The method may comprise causing, with the controller, the light source to increase an intensity of the artificial light by a first percentage; estimating, with the controller, a CO₂ demand of the seaweed biomass responsive to the increased intensity; and modifying, with the controller, the flow of CO₂ responsive to the CO₂ demand. The method may comprise causing, with the controller, the light source to increase the intensity of the artificial light by a second percentage if the CO₂ demand increases responsive to the increased intensity; or decrease the intensity of the artificial light by a third percentage if the CO₂ demand decreases responsive to the increased intensity. The method may comprise causing, with the controller, the light source to decrease the intensity comprises: decreasing the intensity of the artificial light by a predetermined reset amount; waiting for a predetermined period; and repeating steps comprising causing, with the controller, the light source to increase an intensity of the artificial light by the first percentage; estimating, with the controller, a CO₂ demand of the seaweed biomass responsive to the increased intensity; and modifying, with the controller, the flow of CO₂ responsive to the CO₂ demand.

Outputting the sensory data may comprise outputting a temperature of the culture medium. The method may comprise adjusting, with the controller, the at least one of the artificial light or the culture medium responsive to the temperature of the culture medium. The method may comprise modifying the culture medium by exposing the light-transmitting vessel to a temperature regulating fluid in the light-controlling enclosure responsive to the temperature of the culture medium. The method may comprise exposing the light-transmitting vessel to the temperature regulating fluid comprises circulating the temperature regulating fluid around the light-transmitting vessel. The method may comprise exposing the light-transmitting vessel to the temperature regulating fluid comprises causing, with the controller, a pump to circulate an amount of water around the light-transmitting vessel. Exposing the light-transmitting vessel to the temperature regulating fluid comprises causing, with the controller, a temperature regulation unit to output a flow of conditioned air toward the light-transmitting vessel responsive to the temperature of the culture medium.

Intermittently mixing and cutting the seaweed biomass may comprise cutting the seaweed mass with a cutting element sealed in the light-transmitting vessel. A blade may be sealed in the light-transmitting vessel and intermittently mixing and cutting the seaweed biomass may comprise causing, with the controller, a motor to cut the seaweed biomass by rotating the blade in a first direction relative to light-transmitting vessel. Intermittently mixing and cutting the seaweed biomass may comprises causing, with the controller, the motor to mix the seaweed biomass by rotating the blade in a second direction opposite the first direction. The method may comprise causing, with the controller, the motor to continuously rotate the blade in the second direction at a first RPM for a period; and intermittently rotate the blade in the first direction at a second RPM during the period. The second direction may be opposite of the first direction. The second RPM may be greater than the first RPM. Intermittently mixing and cutting the seaweed biomass may comprise causing, with the controller, an aerator to mix the seaweed biomass by aerating the culture medium in the light-transmitting vessel. Removing the portion of the seaweed biomass may comprise pressurizing a head space of the light-transmitting vessel. Pressurizing the head space comprises directing, with the controller, a flow of CO₂ to into the head space. Outputting the sensory data may comprise causing, with the controller, an optical sensor to determine an opacity of the culture medium; and adjusting, with the controller, the at least one of the artificial light or the culture medium responsive to the opacity of the culture medium.

Related seaweed bioreactor apparatus, methods, and systems also are disclosed.

### BRIEF DESCRIPTION OF THE DRAWINGS

The accompanying drawings, which are incorporated in and constitute part of this disclosure, illustrate exemplary aspects that, together with the written descriptions, serve to explain the principles of this disclosure. Numerous aspects are particularly described, pointed out, and taught in the written descriptions. Some structural and operational aspects may be even better understood by referencing the written portions together with the drawings. A more complete appreciation of the subject matter of this disclosure and described advantages may thus be realized by reading the written descriptions set forth herein together with the following accompanying drawings:

FIG. 1 depicts an exemplary seaweed system comprising a plurality of seaweed apparatus;

FIG. 2 depicts an exemplary seaweed apparatus of the FIG. 1 system;

FIG. 3 depicts the FIG. 2 apparatus with a front wall removed;

FIG. 4 depicts exemplary interior elements of the FIG. 2 apparatus;

FIG. 5 depicts exemplary interior elements of the FIG. 2 apparatus;

FIG. 6 depicts an exemplary blade of the FIG. 2 apparatus;

FIG. 7 depicts an installed front view of the FIG. 3 apparatus;

FIG. 8 depicts an installed back view of the FIG. 2 apparatus with a cover removed;

FIG. 9 depicts an exemplary controller for the FIG. 2 apparatus;

FIG. 10 depicts an installed top view of the FIG. 3 apparatus;

FIG. 11 depicts a conceptual schematic for the FIG. 2 apparatus;

FIG. 12 depicts another conceptual schematic for the FIG. 2 apparatus;

FIG. 13 depicts other exemplary blades of the FIG. 2 apparatus;

FIG. 14 depicts another exemplary seaweed system comprising a plurality of seaweed bioreactor apparatus;

FIG. 15 depicts a conceptual schematic for a portion of the FIG. 14 system;

FIG. 16 depicts an exemplary seaweed apparatus of the FIG. 14 system;

FIG. 17 depicts exemplary interior elements of the FIG. 16 apparatus;

FIG. 18 depicts a conceptual schematic for the FIG. 16 apparatus;

FIG. 19 depicts another conceptual schematic for the FIG. 16 apparatus;

FIG. 20 depicts an exemplary valve of the FIG. 16 apparatus;

FIG. 21 depicts an exemplary section view of the FIG. 20 valve;

FIG. 22 depicts another exemplary section view of the FIG. 20 valve;

FIG. 23 depicts alternative exemplary interior elements of the FIG. 16 apparatus;

FIG. 24 depicts a conceptual schematic for the FIG. 23 apparatus;

FIG. 25 depicts another conceptual schematic for the FIG. 23 apparatus;

FIG. 26 depicts another exemplary seaweed system; and

FIG. 27 depicts yet another exemplary bioreactor system.

### DETAILED DESCRIPTION

Aspects of the present disclosure are not limited to the exemplary structural details and component arrangements described in this description and shown in the accompanying drawings. Many aspects of this disclosure may be applicable to other aspects and/or capable of being practiced or carried out in various variants of use, including the examples described herein.

Throughout the written descriptions, specific details are set forth in order to provide a more thorough understanding to persons of ordinary skill in the art. For convenience and ease of description, some well-known elements may be described conceptually to avoid unnecessarily obscuring the focus of this disclosure. In this regard, the written descriptions and drawings should be interpreted as illustrative rather than restrictive, enabling rather than limiting.

Exemplary aspects of this disclosure reference various seaweed bioreactor apparatus, methods, and systems. Some aspects are described with reference to maintaining a particular type of seaweed (e.g., one having a biphasic or haploid-diploid life cycle, such as kelp) utilizing a particular culture medium (e.g., water) that is contained in a particular structure (e.g., an enclosed vessel) and operable with a particular life support system (e.g., one utilizing the various motors, pumps, and sensors described herein) performing a particular set of functions (e.g., managing light and nutrients) optimal for growing the seaweed. Unless claimed, these descriptions are provided for convenience and not intended to limit this disclosure. Accordingly, any aspects described in this disclosure may be utilized with any similar bioreactor apparatus, methods, and systems-including those suitable for use with other types of plants.

Subheadings are provided for ease of reference and non-limiting unless claimed.

Inclusive terms such as “comprises,” “comprising,” “includes,” “including,” and variations thereof, are intended to cover a non-exclusive inclusion, such that any apparatus, method, system, or element thereof comprising a list of elements does not include only those elements but may include other elements not expressly listed and/or inherent thereto. Unless stated otherwise, the term “exemplary” is used in the sense of “example,” rather than “ideal.” Various terms of approximation may be used in this disclosure, including “approximately” and “generally.” Approximately means “roughly” or within 10% of a stated number or outcome. Generally means “usually” or more than a 50% probability.

Terms such as “attachable to,” “attached to,” and “attached” are intended to describe a structural connection between two or more elements. Some structural connections may be “fixedly attached” and thus non-rotatable, as when the two or more elements are formed together and cannot be rotated independently without damage. Other structural connections may be “rotatably attached,” as when the two or more elements are coupled together by attachment elements (e.g., pins, screws, etc.) and/or linking elements (e.g., joints, hinges, etc.) allowing for independent rotation. Unless stated otherwise, the term “attach” and its equivalents may thus comprise any such variations.

Aspects of exemplary controllers are described. The controllers may comprise and be operable with any type of software and/or hardware. Functional terms such as “processing,” “computing,” “calculating,” “determining,” “displaying,” and the like, may refer to actions and processes performable by the controller.

The software may comprise program objects (e.g., blocks of codes) executable by a controller to perform functions. Each program object may comprise a sequence of operations leading to a desired result, like an algorithm. The operations may require or involve physical manipulations of physical quantities, such as electrical or magnetic signals capable of being stored, transferred, combined, compared, and otherwise manipulated. The signals may be described conceptually as bits, characters, elements, numbers, symbols, terms, values, or the like.

The hardware may comprise any known computing and/or networking devices that are specially or generally configured to execute the program objects, perform the operations, and/or send or receive the signals. The hardware may comprise a processor that executes the project objects by manipulating and/or transforming input data represented as physical (electronic) quantities within the unit's registers and memories into output data similarly represented as physical quantities within the unit's memories or registers and/or other data storage, transmission, or display devices. The processor may comprise any number of processing element(s), including any singular or plural computing resources disposed local to or remote from one another. The program objects may be stored in any machine (e.g., computer) readable storage medium in communication with the processing unit, including any mechanism for storing or transmitting data and information in a form readable by a machine (e.g., a computer). Exemplary storage mediums may comprise read only memory (“ROM”); random access memory (“RAM”); erasable programmable ROMs (“EPROMs”); electrically erasable programmable ROMs (“EEPROMs”); magnetic or optical cards or disks; flash memory devices; and/or any electrical, optical, acoustical, or other form of propagated signals (e.g., carrier waves, infrared signals, digital signals, etc.).

Some functions are described with reference to method steps performable with the controller. The steps may define an exemplary sequence of operation, the order of which may be important. For example, a particular order of any method steps may describe a particular sequence of operation that is performable by the controller to realize specific processing benefits, such as improving a computational performance and/or an operational efficiency of the controller.

### Examples of a Seaweed System Optimized for Seaweed Biomasses

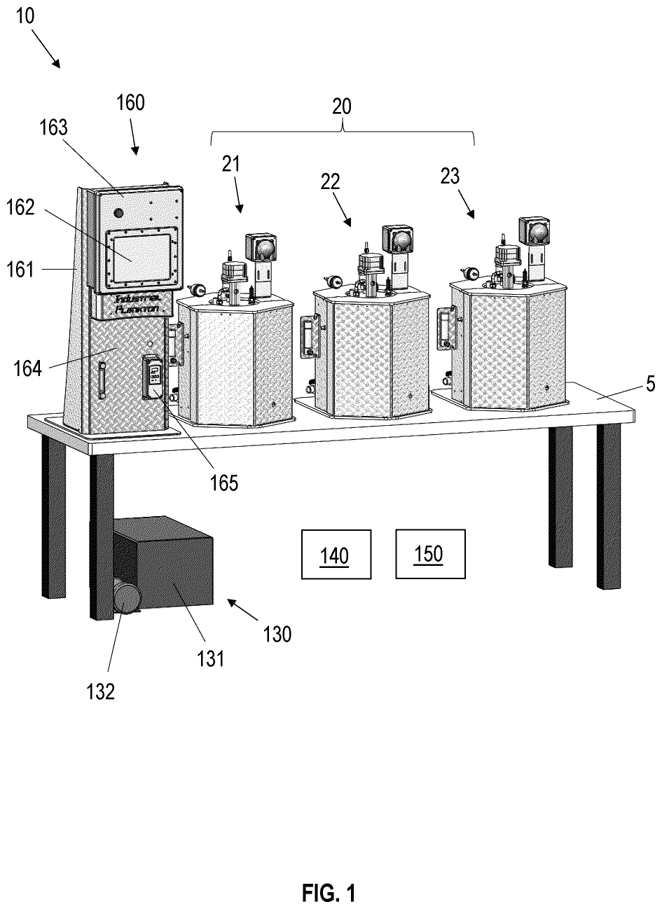

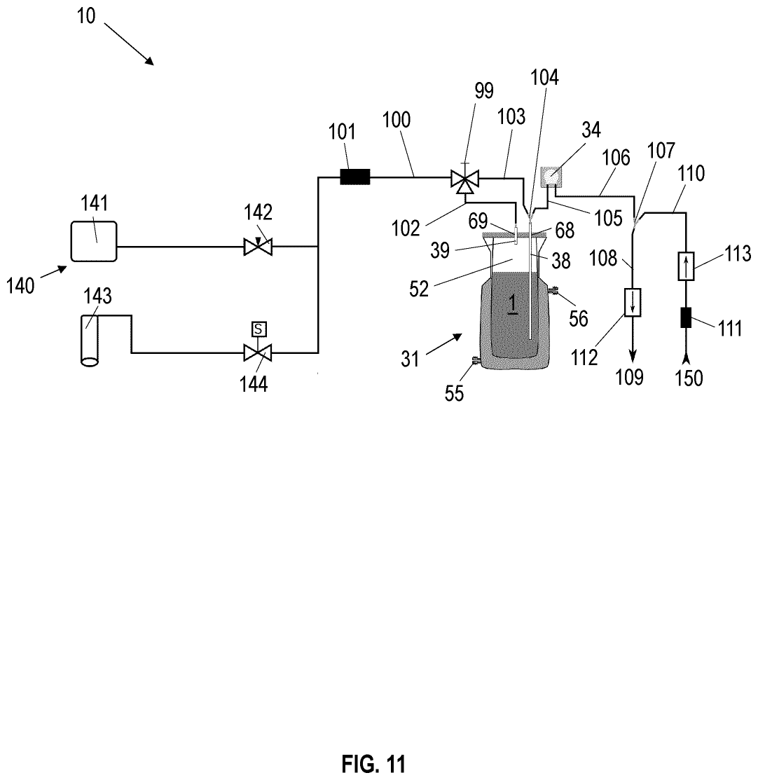

Aspects of this disclosure are now described with reference to an exemplary seaweed bioreactor system 10 for maintaining optimal conditions for growing seaweed in a culture medium 1 (e.g., as shown in FIG. 11), such as fresh water, saltwater, and anything added thereto. Numerous configurations of system 10 are described herein. As shown in FIG. 1, for example, system 10 may comprise a seaweed bioreactor 20; a temperature control system 130; a CO₂ and/or air supply 140; a culture medium supply 150; and a system controller 160.

As shown in FIG. 1, for example, seaweed bioreactor 20 may comprise a plurality of operating elements including a seaweed bioreactor apparatus 21, a seaweed bioreactor apparatus 22, and a seaweed bioreactor apparatus 23. Bioreactor apparatus 21, 22, and 23 may be similar and/or identical to one another. Each apparatus 21, 22, and 23 may serve a different function in system 10. For example: bioreactor apparatus 21 may contain and be optimized to grow a first culture of seaweed cells in a first volume of culture medium 1, such as male gametophytes; bioreactor apparatus 22 may contain and be optimized to grow a second culture of seaweed cells in a second volume of culture medium 1, such as female gametophytes; and bioreactor apparatus 23 may receive and combine the first and second cultures in a third volume of culture medium 1, allowing them to join and become sporophytes (or kelp seedlings). When equipped with seaweed bioreactor apparatus 21, 22, and 23, system 10 may be operable with system controller 160 to maintain optimal conditions for growing each different type of seaweed cell, making it possible to reliably output a steady supply of gametophytes and/or sporophytes.

As shown in FIG. 1, for example, each seaweed bioreactor apparatus 20 may be located on a support structure 5, such as a table. Any type of support structure 5 may be utilized.

As shown in FIG. 1, system 10 may comprise any number of seaweed bioreactor apparatus (e.g., like apparatus 21, 22, and/or 23) operable to increase the number of gametophytes and/or sporophytes output by system 10. Seaweed bioreactor 20 may be utilized to crossbreed different types of seaweed species in a controlled environment with repeatable results. As shown in FIG. 1, for example, seaweed bioreactor apparatus 21 and 22 may contain first and second cells of a first species of seaweed and be in fluid communication with at least one bioreactor apparatus 23. System 10 may comprise any number of additional sets of bioreactor apparatus like 21 and 22, each of which may contain first and second cells for a different species of seaweed and be in fluid communication with at least one bioreactor apparatus 23. In this example, bioreactor apparatus 23 may receive and combine the first (e.g., male gametophytes) and second seaweed cells (e.g., female gametophytes) of the different species of seaweed, thus providing an easier and more efficient means of crossbreeding.

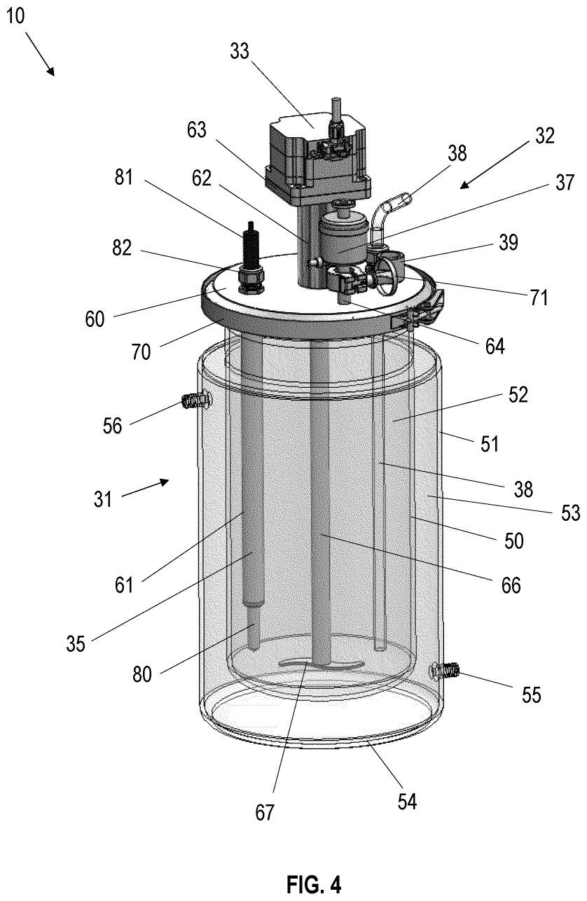

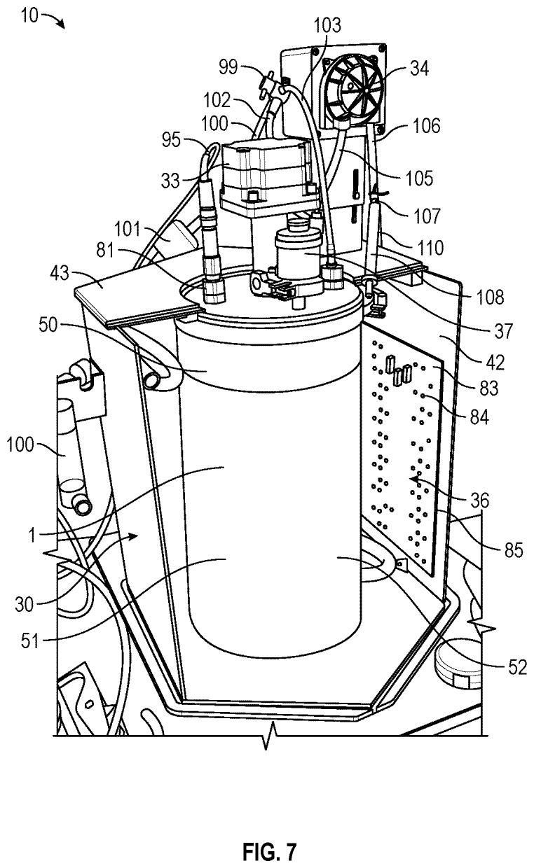

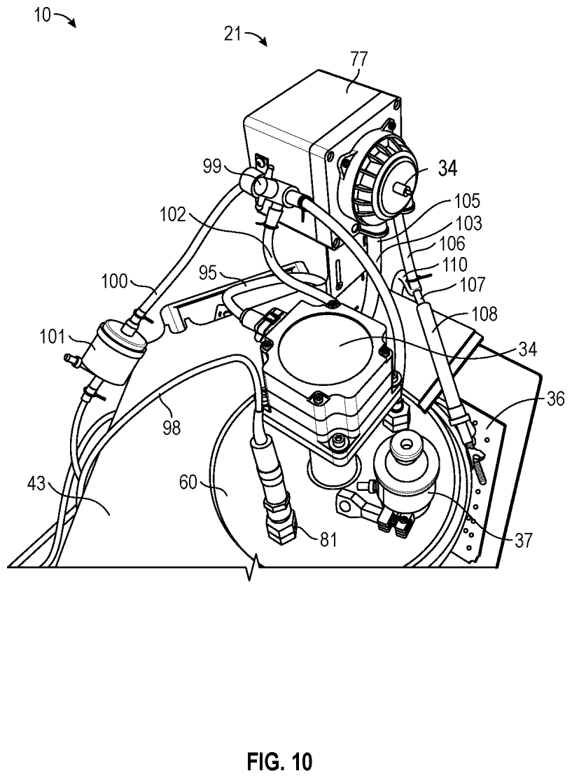

Elements of seaweed bioreactor 20 may be in fluid communication with one another and temperature control system 130, CO₂ and/or air supply 140, and culture medium supply 150 utilizing any type of tubing and/or piping, including the exemplary configurations shown in FIGS. 7-12 and described in relation thereto. As shown in FIG. 1, for example, temperature control system 130 may comprise a chiller 131 and a pump 132. Chiller 131 may store a cooling medium (e.g., a volume of water) and maintain a temperature of the cooling medium. Pump 132 may comprise a centrifugal pump operable to circulate the cooling medium throughout system 10. As shown in FIG. 1, for example, pump 132 may be operable with system controller 160 to circulate the cooling medium from chiller 131, around a thermal jacket of each seaweed bioreactor 20 (e.g., as shown in FIG. 4 and described below), and back to chiller 131 at a flow rate that maintains a temperature of culture medium 1 contained in each bioreactor 20.

System controller 160 may comprise any combination of hardware and/or software operable to maintain optimal conditions for growing seaweed in each bioreactor 20 of system 10. As shown in FIG. 1, for example, system controller 160 may comprise a housing 161, a display element 162, a processing element 163, a communication bus 164, and a temperature controller 165. Housing 161 may comprise a free-standing structure made of a rigid material. As shown in FIG. 1, for example, the rigid material may comprise a metallic material (e.g., diamond plate) operable to support and/or contain the elements of system controller 160 on support structure 5 and act as a heat sink for those elements. Processing element 163 may be located in an upper compartment of housing 161 and comprise data processing elements operable to receive data from and send control signals to other elements of system 10. For example, processing element 163 may comprise one or more of a processor(s), a memory, and/or a transceiver operable with software (e.g., stored on the memory) to receive and process the data and/or generate and output the control signals.

Display element 162 may comprise a touchscreen display operable with processing element 163 to present a graphical user interface to a user and receive touch-based inputs from that user. As shown in FIG. 1, for example, communication bus 164 may be in a lower compartment of housing 161 and comprise any wired and/or wireless data communication hardware for establishing and maintaining data connection(s) operable to receive data from a microcontroller of each seaweed bioreactor 20, relay control signals back to each microcontroller, and send or receive other types of data.

Temperature controller 165 may comprise any combination of hardware and/or software operable with temperature control system 130 to receive data from processing element 163 and/or the microcontrollers and modify the temperature and/or flow rate of the cooling medium circulating through system 10 based on the data.

As shown in FIG. 11, for example, CO₂ and/or air supply 140 may comprise an air pump 141 operable with an air control valve 142 such as a manually operated valve; and a CO₂ supply (e.g., such as CO₂ tank) 143 operable with a CO₂ supply valve 144, such as a solenoid valve operable with system controller 160 (e.g., as shown in FIG. 1). As shown in FIG. 11, culture medium supply 150 may comprise a water line.

Although proximate to seaweed bioreactor(s) 20 in FIG. 1, all or portions of system controller 160 may be located remotely from system 10. As described further below, for example, each seaweed bioreactor 20 may comprise a microcontroller operable to handle a portion of the data processing and/or control signal generating locally, meaning that all or a portion of system controller 160 may be located in the cloud and operable to receive the data and/or output the control signals over the internet. In keeping with this example, a cloud-based system controller 160 may thus be operable with the microcontroller of seaweed bioreactors 20 located at different physical locations, making it easier and/or less expensive to acquire and operate system 10 in an automated fashion.

### Examples of Seaweed Bioreactor Apparatus and Systems

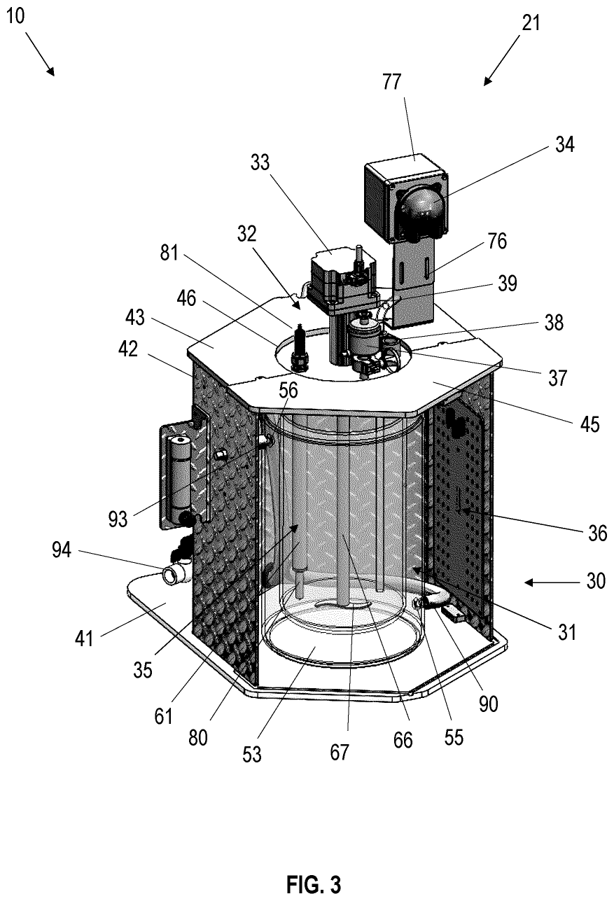

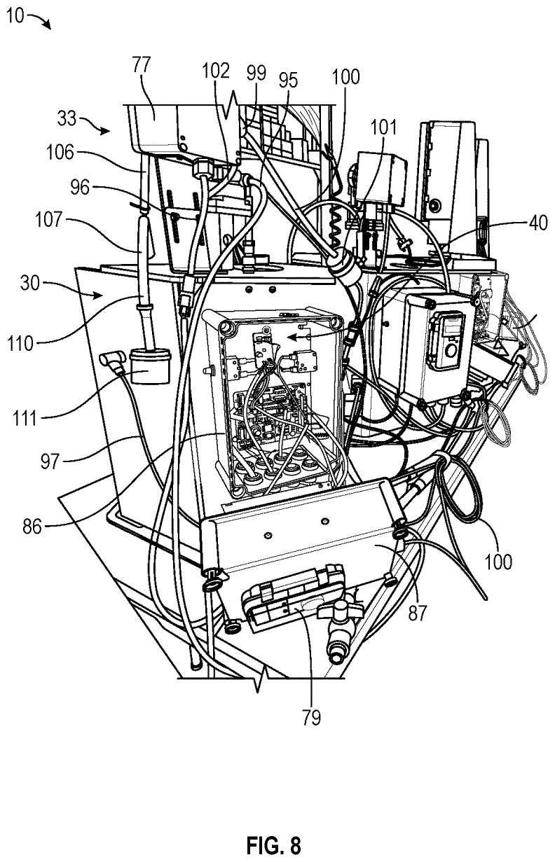

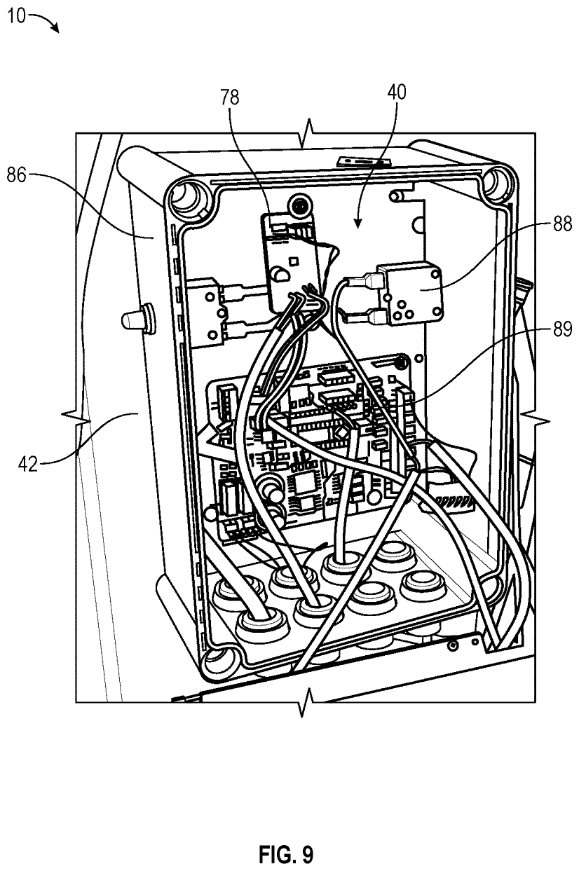

Aspects of this disclosure are now described with reference to an exemplary seaweed bioreactor apparatus 21 operable to maintain optimal conditions for growing seaweed cells in a volume of culture medium 1. These aspects are described with reference to seaweed bioreactor apparatus 21 of FIG. 1 but could alternatively be described with reference to bioreactor apparatus 22 and/or 23 of FIG. 1. Numerous potential configurations of seaweed bioreactor apparatus 21 are described. As shown in FIG. 3, for example, seaweed bioreactor apparatus 21 may comprise a light-tight enclosure 30, a housing 31, a mounting assembly 32, a motor 33, a pump 34, a culture medium sensor 35, a light source 36, a vent filter 37, a port 38, a port 39, and a microcontroller 40 (e.g., as shown in FIGS. 8 and 9).

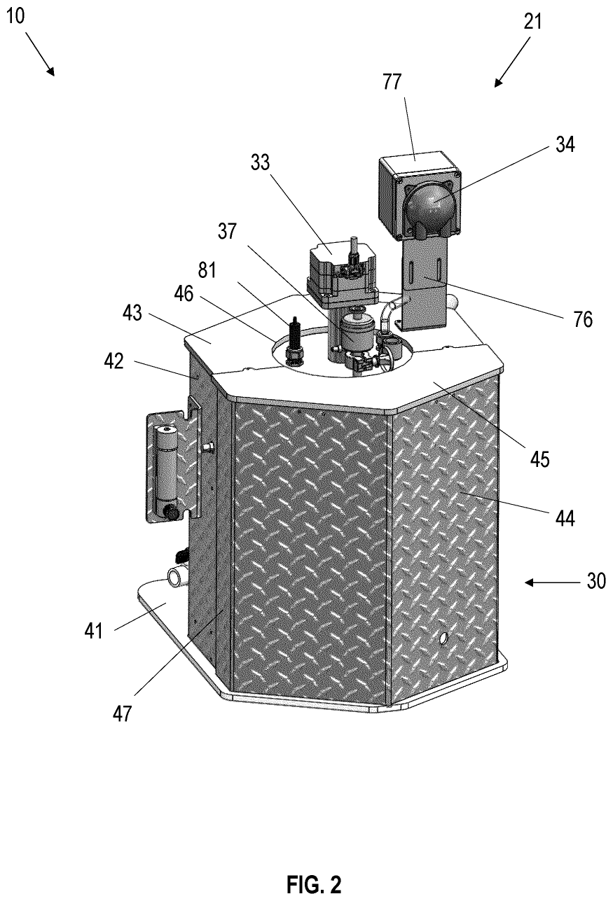

As shown in FIGS. 2 and 3, for example, light-tight enclosure 30 may be made of a rigid material that seals off housing 31 from ambient light, making it possible to carefully control the light spectrum applied to any seaweed cells that are contained in a volume of culture medium 1 sealed in housing 31. Like housing 161 of system controller 160, the rigid material of light-tight enclosure 30 may comprise a metallic material (e.g., diamond plate) operable to support and/or contain element(s) of seaweed bioreactor apparatus 21 and act as a heat sink for those element(s). As shown in FIG. 2, for example, light-tight enclosure 30 may comprise a floor plate 41, a rear wall 42, a rear top plate 43, a front wall 44, a front top plate 45, and a receiving opening 46.

Rear wall 42 may be attached to floor plate 41 and rear top plate 43 to create a rigid structure operable to support the weight of seaweed bioreactor apparatus 21 on support surface 5 (e.g., FIG. 1). As shown in FIGS. 2 and/or 3, for example, a top surface of floor plate 41 and a bottom surface of rear top plate 43 may comprise grooves sized to receive corresponding top and bottom edges of rear wall 42, thereby forming the rigid structure. Front wall 44 may be attached to the rigid support structure to prevent ambient light from reaching housing 31 through the sides of enclosure 30. As shown in FIG. 2, for example, front wall 44 may comprise rear portions 47 that overlap front portions of rear wall 42 when front wall 44 is attached, thereby preventing the ambient light from reaching housing 31 through the sides of enclosure 30. As shown in FIG. 3, for example, the groove of floor plate 41 may be sized to minimize light entry by maintaining interior surfaces of rear portions 47 against exterior surfaces of rear wall 42.

Front top plate 45 also may be attached to front wall 44 and rear top plate 43 to prevent the ambient light from reaching housing 31 through the top of enclosure 30. As shown in FIG. 3, for example, a bottom surface of front top plate 45 may comprise a groove sized to receive top edges of rear wall 42, front wall 44, and rear portions 47 to further maintain interior surfaces of rear portions 47 against exterior surfaces of rear wall 42. As shown in FIGS. 3 and 4, for example, a front edge of rear top plate 43 may interlock with a rear edge of front top plate 45 to prevent the ambient light from reaching housing 31 between the front and rear edges of plates 43 and 45. Additional light sealing elements may be utilized to further seal enclosure 30, such as an expandable foam and/or light-blocking tape. As shown in FIG. 2, for example, receiving opening 46 may be formed when rear plate 43 is interlocked with front plate 45.

Housing 31 may be light transmitting, meaning made of a transparent material or a translucent material. As shown in FIG. 4, for example, housing 31 may comprise an inner wall 50 and an outer wall 51. Inner wall 50 may define a vessel 52 sized to contain a volume of culture medium 1 containing an amount of seaweed cells. Accordingly, both of housing 31 and vessel 52 also may be described as a light transmitting vessel.

As shown in FIG. 4, for example, vessel 52 may comprise a cylindrical shape with an open top and a closed bottom. Inner wall 50 may be spaced apart from outer wall 51 to define a thermal jacket 53 that at least partially surrounds vessel 52. As shown in FIG. 4, for example, outer wall 51 may comprise a base 54, an inlet 55, and an outlet 56. Base 54 may be placed on top of floor plate 41 (e.g., as shown in FIGS. 2 and 3) and operable to support the weight of vessel 52 and its contents thereon. Inlet 55 and outlet 56 may be placed in fluid communication with chiller 131 and operable with pump 132 to direct the cooling medium into and out of thermal jacket 53 at a flow rate determined by temperature controller 165. As described further below, each of inner wall 50, outer wall 51, and the cooling medium may be generally translucent.

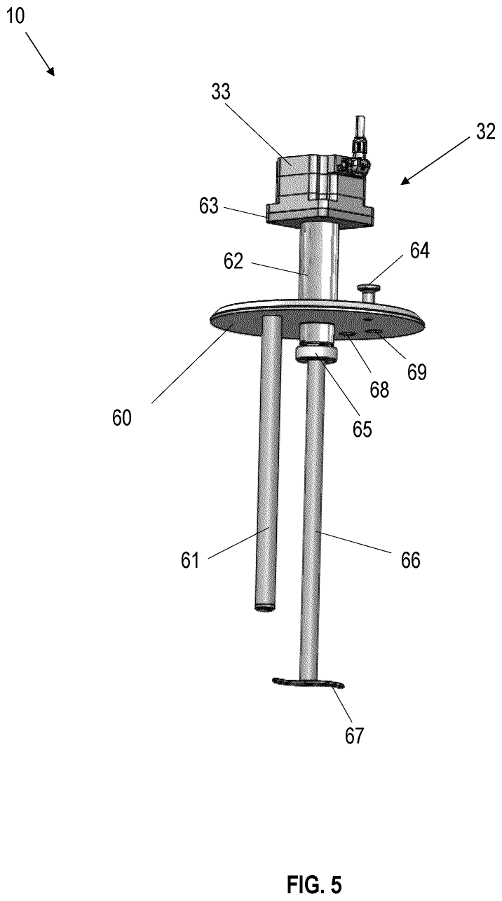

As shown in FIGS. 4 and/or 5, for example, mounting assembly 32 may comprise a mounting plate 60, a sensor support tube 61, a bearing shaft 62, a motor mount 63, a filter mount 64, a driveshaft coupler 65, a drive shaft 66, a blade 67, a hole 68, and a hole 69. As shown in FIG. 4, for example, mounting plate 60 may be removably attached to the top of inner wall 50 and operable to seal vessel 52. As shown in FIG. 4, for example, a rim seal 70 may be utilized to form an airtight seal between the bottom of mounting plate 60 and top surfaces of vessel 52 that prevents ambient air from entering vessel 52. Sensor support tube 61 may comprise a hollow tube that is attached to and extends through mounting plate 60 toward a bottom portion of vessel 52. Bearing shaft 62 may comprise a hollow tube that is attached to and extends through mounting plate 60 toward a top portion of vessel 52. Motor mount 63 may be fixedly attached to a top of bearing shaft and comprise an opening extending therethrough. As shown in FIGS. 4 and 5, for example, motor 33 may be attached to motor mount 63 to form an airtight seal between the bottom of motor 33 and the top of motor mount 63 that prevents ambient air from entering vessel 52. An output shaft of motor 33 may be directed through the opening of mount 63.

Vent filter 37 may prevent unfiltered ambient air from entering vessel 52 if a vacuum is formed when operating pump 34 and/or performing any method described. As shown in FIGS. 4 and 5, for example, filter mount 64 may comprise a hollow tube that is attached to and extends through mounting plate 60. Vent filter 37 may be removably attached to a flange of filter mount 64 with a valve 71 operable to form an airtight seal between vent filter 37 and filter mount 64. As shown in FIG. 5, for example, drive shaft coupler 65 may comprise a rotational bearing that attaches the output shaft of motor 33 with drive shaft 66. Drive shaft coupler 65 may form an airtight seal between the bottom of bearing shaft 62 and the top of drive shaft coupler 65 that prevents ambient air from entering vessel 52. Drive shaft 66 may be attached to drive shaft coupler 65 and blade 67.

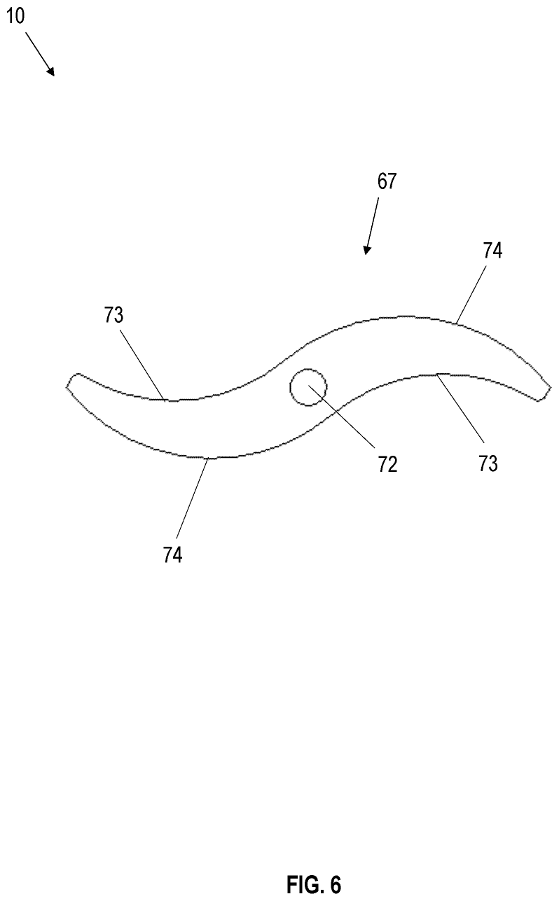

As shown in FIG. 6, for example, blade 67 may comprise a hole 72, a pair of shredding or sickle-shaped edges 73, and a pair of mixing or scimitar-shaped edges 74. Hole 72 may be sized to receive a bolt that attaches drive shaft 66 to blade 67. As described further below, each shredding or sickle-shaped edge 73 may comprise a concave blade or edge operable to shred the contents of vessel 52 when drive shaft 66 is rotated in a first or “shredding” direction (e.g., counter-clockwise) by motor 33 and each mixing or scimitar-shaped edge 74 may comprise a convex blade or edge operable to mix the contents of vessel 52 when drive shaft 66 is rotated in a second or “mixing” direction (e.g., clockwise) by motor 33.

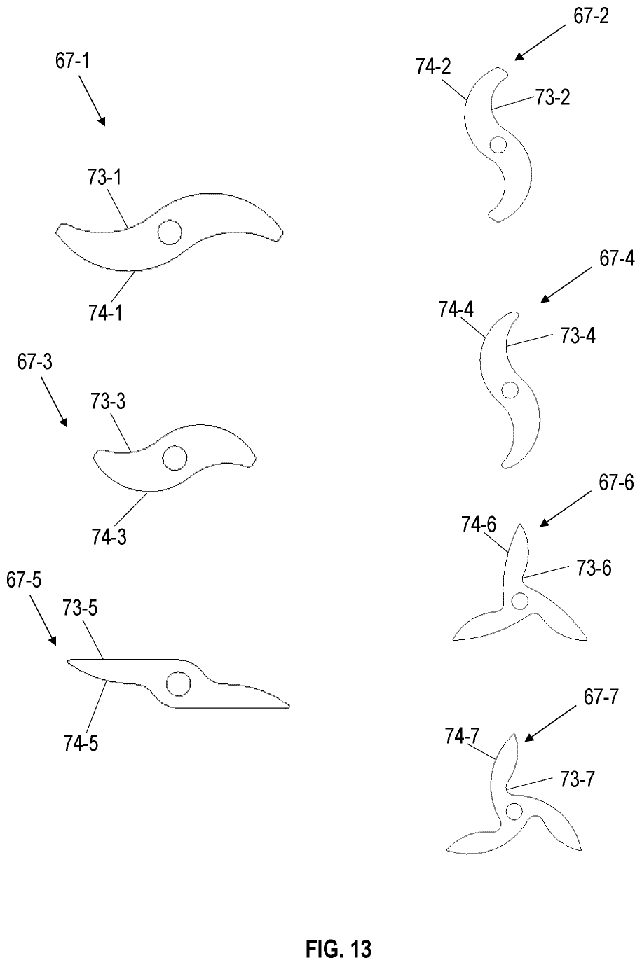

To provide additional examples, seven variations of blade 67 are shown in FIG. 13 and labeled as blades 67-1, 67-2, 67-3, 67-4, 67-5, 67-6, and 67-7, each of which, like blade 67, may comprise at least one pair of shredding or sickle-shaped edges 73-1, 73-2, 73-3, 73-4, 73-4, 73-5, 73-6, or 73-7 operable to shred the contents of vessel 52 when drive shaft 66 is rotated in the shredding direction by motor 33 (e.g., a counter-clockwise direction) and at least one pair of mixing or scimitar-shaped edges 74-1, 74-2, 74-3, 74-4, 74-5, 74-6, or 74-7 operable to mix the contents of vessel 52 when drive shaft 66 is rotated in the mixing direction by motor 33 (e.g., a clockwise direction).

As shown in FIGS. 4 and/or 5, for example, port 38 may comprise a translucent hollow tube with a top end that may be placed in fluid communication with pump 34 (e.g., as shown in FIGS. 2 and 3) and a bottom end that extends through hole 68 toward a bottom portion of vessel 52. As also shown in FIGS. 4 and/or 5, for example, port 39 may comprise a translucent hollow tube with a top end that may be placed in fluid communication with CO₂ and/or air supply 140 (e.g., as shown in FIGS. 1, 11, and 12) and a bottom that extends through hole 69 toward an interior of vessel 52. Pump 34 may be operable with microcontroller 40 and/or system controller 160 to output a flow rate of culture medium 1 and seaweed cells from vessel 52 or input a flow rate of new culture medium 1 into vessel 52. As shown in FIGS. 2 and 3, for example, pump 34 may comprise a peristaltic pump with all wetted components such as a Welco WP11. As shown in FIGS. 2 and 3, for example, pump 34 may comprise a pump mount 76 and a pump box 77. Pump mount 76 may attach pump 34 to rear top plate 43 and pump box 77 may contain an electric motor operable with microcontroller 40 and/or system controller 160.

Culture medium sensor 35 (e.g., as shown in FIG. 4) may be operable with microcontroller 40 and/or system controller 160 to output data associated with a characteristic of culture medium 1 in vessel 52. Culture medium sensor 35 may comprise a sensing end 80 and a communication end 81. A shown in FIG. 4, for example, sensor 35 may be receivable in sensor support tube 61 so that sensing end 80 is in a bottom portion of vessel 52 and communication end 81 is outside of vessel 52. Sensing end 80 may comprise a pH sensor operable to output data associated with a pH level of culture medium 1 and/or a temperature sensor operable to output data associated with a temperature of culture medium. Communication end 81 may comprise a power/data connection, such as a 4 pin, military-style analog connection. As shown in FIG. 4, for example, communication end 81 also may comprise a threaded connection 82 operable to form an airtight seal that prevents ambient air from entering vessel 52 when sensor 35 is received in sensor support tube 61.

As shown in FIGS. 3 and 7, for example, light source 36 may comprise a printed circuit board or “PCB” 83, a plurality of LEDs 84, and a generally translucent panel 85. As shown in FIG. 7, for example, PCB 83 may be mounted to an interior surface of rear wall 42 of enclosure 40. Plurality of LEDs 84 may comprise different clusters and/or strips of spectrum selectable LEDS (e.g., such as RGB LEDs or “red, blue and green LEDs”) that are mounted to a vessel-facing side of PCB 83 and operable with microcontroller 40 and/or system controller 160 to direct over 16 million hues of light toward housing 31 (e.g., as shown in FIGS. 4 and 11). Plurality of LEDs 84 may comprise at least one strip of red LEDs that are similarly oriented and operable to direct a red light toward housing 31. As shown in FIG. 7, for example, a wall-facing side of PCB 83 may be thermally coupled to rear wall 42 so that a portion of the heat output from LEDs 84 may be radiated toward wall 42, allowing it to act as a heat sink for LEDs 84. As also shown in FIGS. 2, 3, and/or 7, for example, generally translucent panel 85 (e.g., such as a plexiglass panel) may be mounted to rear wall 42 and positioned in front of LEDs 84 to protect them from damage when moving housing 31 relative to light-tight enclosure 30.

Microcontroller 40 may be contained in a housing 86 having a cover 87. As shown in FIGS. 8 and 9, for example, housing 86 may comprise a plastic enclosure that is mounted to a rear surface of rear wall 42 of light-tight enclosure 30. Cover 87 may be bolted to housing 86. Microcontroller 40 may comprise any combination of hardware and/or software operable with motor 33, pump 34, system controller 160, and/or any other element of system 10 to maintain optimal conditions for growing seaweed in the volume of culture medium 1 contained in vessel 52. As shown in FIGS. 8 and/or 9, for example, microcontroller 40 may comprise a pump signal communicator 78, a motor drive 79, resettable fuses 88, and/or a PCB 89. The circuitry for pump signal communicator 78 and/or motor driver 79 may be separate from PCB 89 and/or integrated into PCB 89.

Examples of Seaweed Systems with Independent Life Support Systems

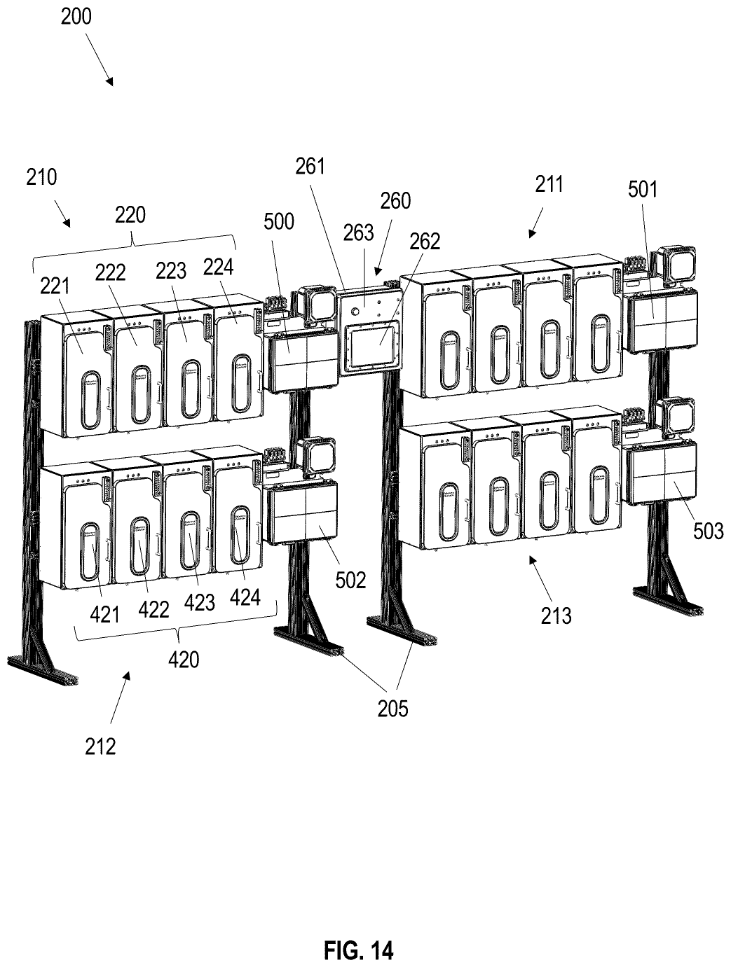

Aspects of this disclosure are now described with reference to an exemplary seaweed system 200 comprising a plurality of individual seaweed bioreactor systems with one or more seaweed bioreactors that are independently operable with a system controller 260 to maintain optimal conditions for growing different seaweed biomasses in one or more separated volumes of culture medium 1. Numerous configurations of seaweed system 200 are described herein, including those described in FIGS. 14, 26, and 27, for example, with reference to individual seaweed bioreactor systems 210, 211, 212, and 213. Aspects of each seaweed bioreactor system 210, 211, 212, and/or 213 may be like aspects of seaweed bioreactor system 10 described above with reference to FIG. 1, for example, but within the 200 series of numbers. Aspects described with reference to one of systems 10, 200, 210, 211, 212, and/or 213 may be interchangeable with any aspects described with reference to another system 10, 200, 210, 211, 212, and/or 213, each iteration and permutation being part of this disclosure.

Within seaweed system 200, each seaweed bioreactor system 210, 211, 212, and 213 may comprise at least one seaweed bioreactor and at least one life support system operable with system controller 260 to maintain optimal conditions for growing seaweed in the seaweed bioreactor. As shown in FIG. 14, for example, each seaweed bioreactor system 200, 210, 211, 212, and 213 along with its respective life support system 500, 501, 502, and 503 may be attached to a support structure 205 (e.g., a rigid metal frame) with system controller 260 so that system 200 becomes an integrated unit.

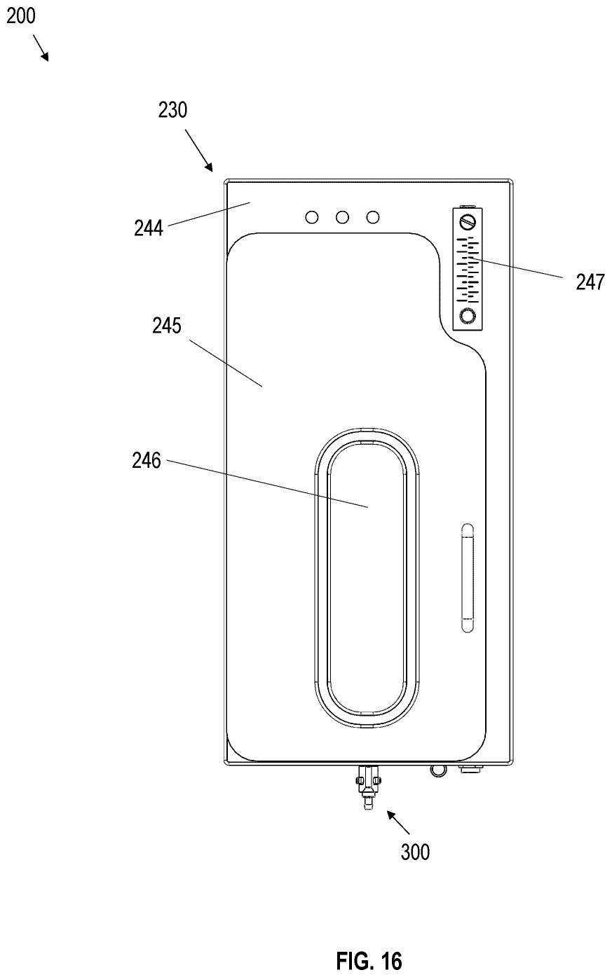

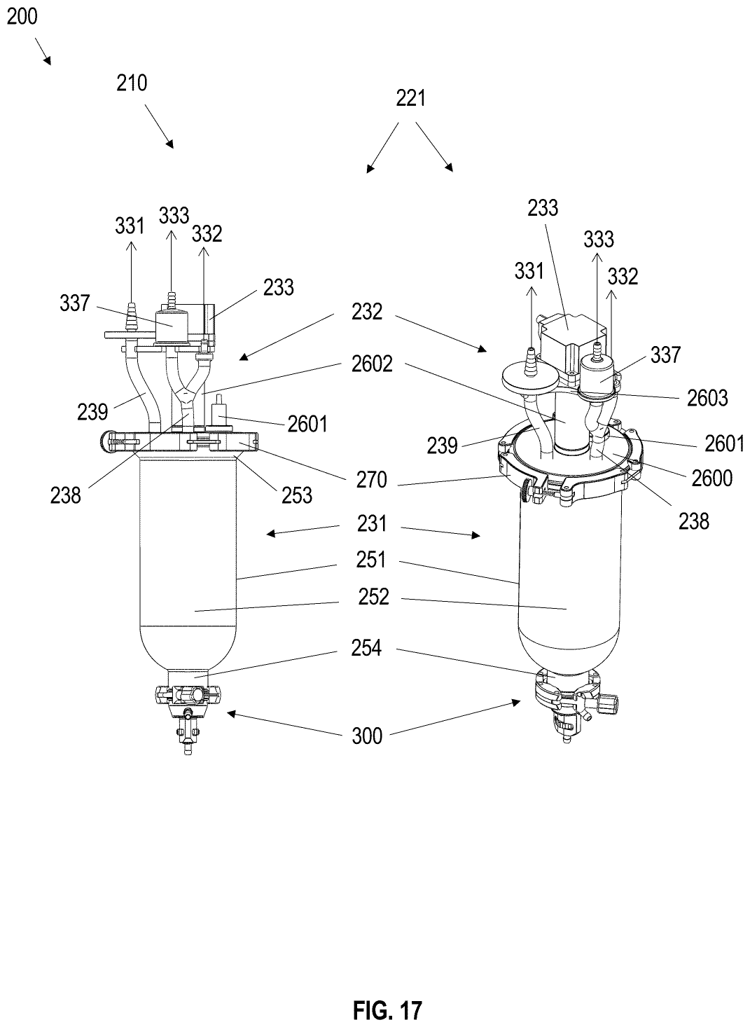

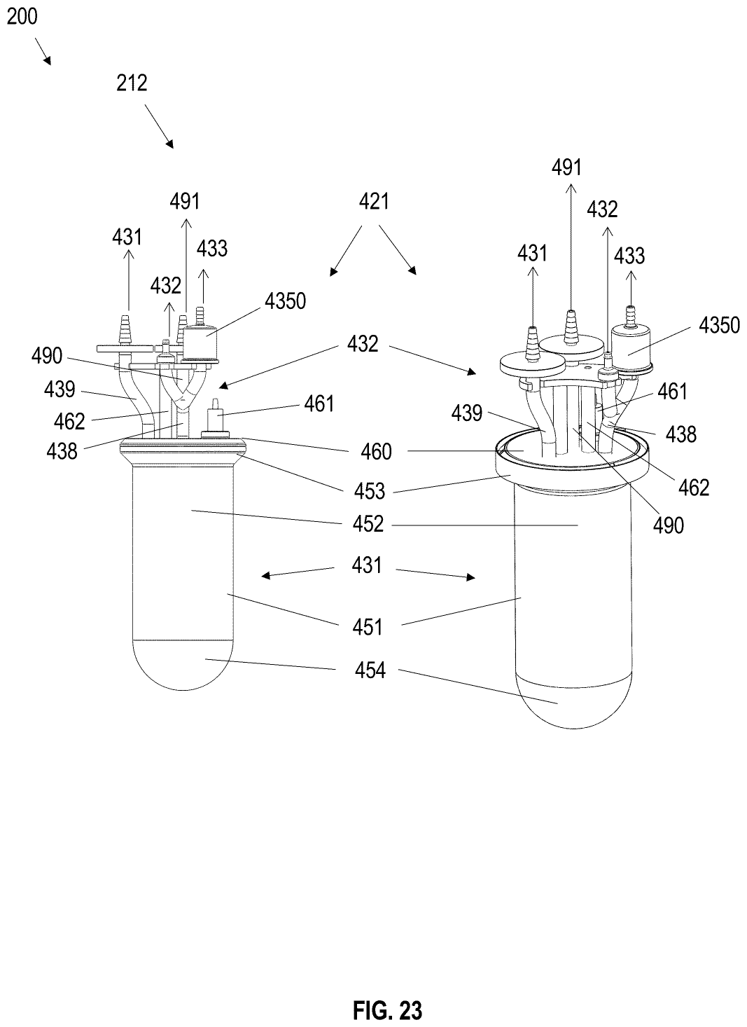

Different types of seaweed bioreactors are described herein, including seaweed bioreactor 20 described above with reference to FIG. 1 and seaweed bioreactors 220, 420 described below with reference to FIG. 14. Each seaweed bioreactor system 210, 211, 212, and 213 may comprise at least one seaweed bioreactor apparatus operable to contain a separated volume of culture medium 1 and maintain optimal conditions for growing a seaweed biomass in the separated volume 1 for extended periods of time, such as months or years. By way of example, aspects of each seaweed bioreactor apparatus in system 210, 211, 212, and/or 213 may be like seaweed bioreactor apparatus 21 described above with reference to FIGS. 2 and 3, for example; like seaweed bioreactor apparatus 221 described below with reference to FIGS. 16 and 17, for example; and/or like seaweed bioreactor apparatus 421 described below with reference to FIGS. 16 and 23, for example, each iteration and permutation being part of this disclosure along with those of 10, 200, 210, 211, 212, and/or 213.

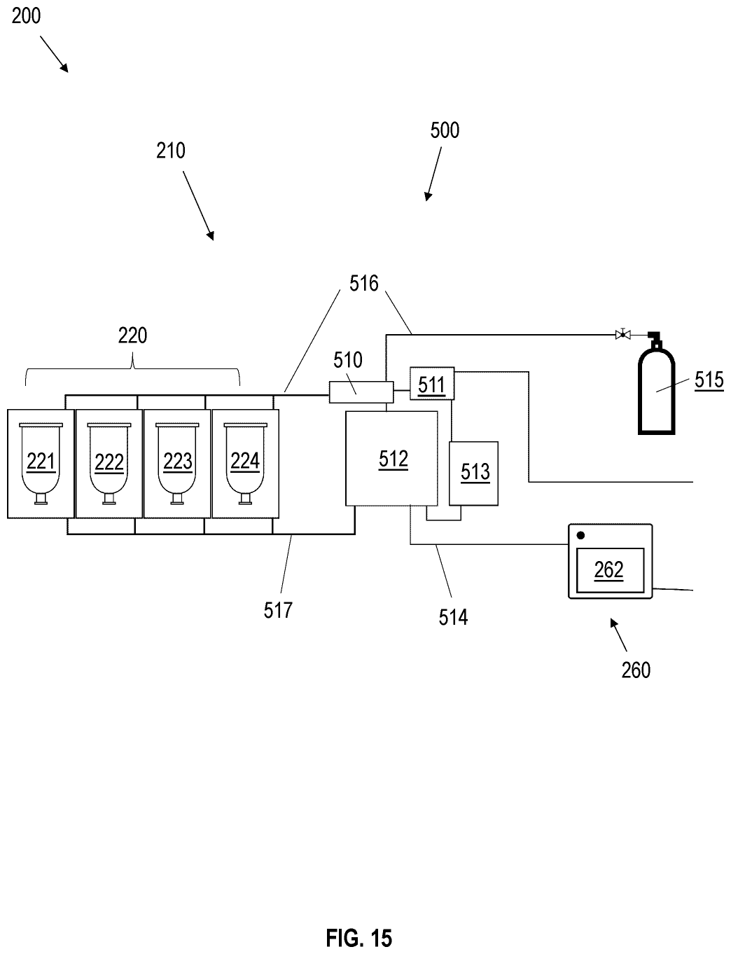

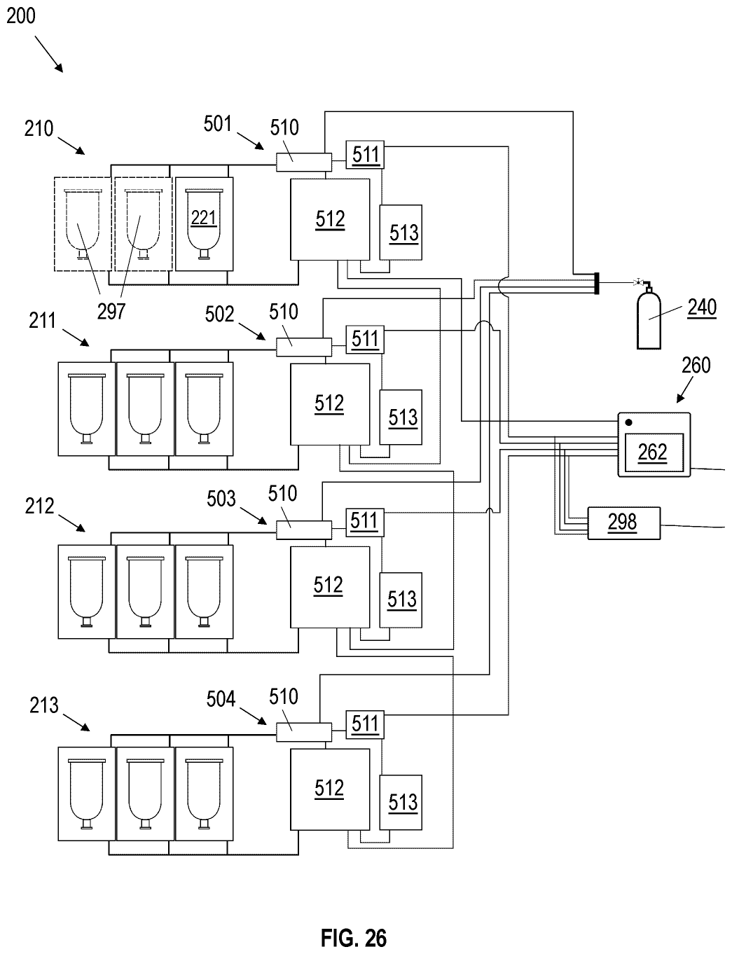

An example of seaweed bioreactor 220 with one seaweed bioreactor apparatus 221 is shown at top left in FIG. 26, for example, in which two expansion slots 298 are drawn with dotted lines to indicate that two more seaweed bioreactor apparatus may be added. As shown in FIGS. 14 and 15, for example, seaweed bioreactor 220, like seaweed bioreactor 20, also may comprise a plurality of seaweed bioreactor apparatus including a seaweed bioreactor apparatus 221, a seaweed bioreactor apparatus 222, a seaweed bioreactor apparatus 223, and/or a seaweed bioreactor apparatus 224. In this example, bioreactor apparatus 221, 222, 223, and 223 may be similar and/or identical to one another, depending upon whether seaweed system 200 is being optimized for the purpose of crossbreeding of different seaweed species or mass producing one seaweed species.

As shown in FIGS. 14 and 15, for example, each seaweed bioreactor apparatus 221, 222, 223, and 224 of seaweed bioreactor 220 may function differently depending on the purpose of seaweed system 200 and the role of seaweed bioreactor system 210 within system 200. In a crossbreeding scenario, for example, bioreactor apparatus 221 may contain and be optimized to grow a first culture of seaweed cells in a first volume of culture medium 1, such as a first species of male gametophytes; bioreactor apparatus 222 may contain and be optimized to grow a second culture of seaweed cells in a second volume of culture medium 1, such as a second species of male gametophytes; bioreactor apparatus 223, may contain and be optimized to grow a third culture of seaweed cells in a third volume of culture medium 1, such as a species of female gametophytes that is compatible with the first and/or second species of male gametophytes; and bioreactor apparatus 224 may receive and combine one of the first or second cultures with the third culture in a fourth volume of culture medium 1, allowing them to join and become different species of sporophytes (or kelp seedlings). In this scenario, seaweed bioreactor systems 210, 211, 212, and 213 may be independently operable with system controller 260 and their respective life support systems 500, 501, 502, and 503 to maintain optimal conditions for growing the different species and/or types of seaweed cells and guiding their reproduction, making it possible to efficiently create new crossbreeds or output known crossbreeds of gametophytes and sporophytes.

As shown in FIGS. 14 and 15, for example, the seaweed bioreactor(s) of each seaweed system 210, 211, 212, and 213 also may play different roles within seaweed system 200 depending on its purpose. In a mass production scenario, for example, the top-level seaweed bioreactors of seaweed systems 210 and 211 may be like seaweed bioreactor 221 and thus optimized to grow male gametophytes in first volumes of culture medium 1 and female gametophytes in second volumes of culture medium 1; and the bottom-level seaweed bioreactors of seaweed systems 212 and 213 may be like seaweed bioreactor 421 and thus optimized receive and combine the male and female gametophytes in third volumes of culture medium 1, allowing them to efficiently join and become sporophytes (or kelp seedlings) of the same species. In this scenario, seaweed bioreactor systems 210, 211, 212, and 213 may again be independently operable with system controller 260 and their respective life support systems 500, 501, 502, and 503 to maintain optimal conditions for growing the different species and/or types of seaweed cells and guiding their reproduction, making it possible to efficiently output gametophytes and sporophytes of a particular breed of seaweed.

In either the crossbreeding or mass production scenarios, seaweed system 200 may comprise any configuration of tubing and related connections for placing seaweed bioreactor systems 210, 211, 212, and 213 or elements thereof in fluid communication with one another. In the crossbreeding scenario, for example, system 200 may comprise tubing and connections for placing seaweed bioreactor apparatus 221, 222, and 223 in fluid communication with seaweed bioreactor apparatus 224. In the mass production scenario, for example, system 200 may comprise tubing and connections for placing the top-level systems 210, 211 in fluid communication with the bottom-level systems 212, 213 so that the top-level systems may be gravity fed to the bottom-level systems (e.g., via diffusing nozzles 300 described below).

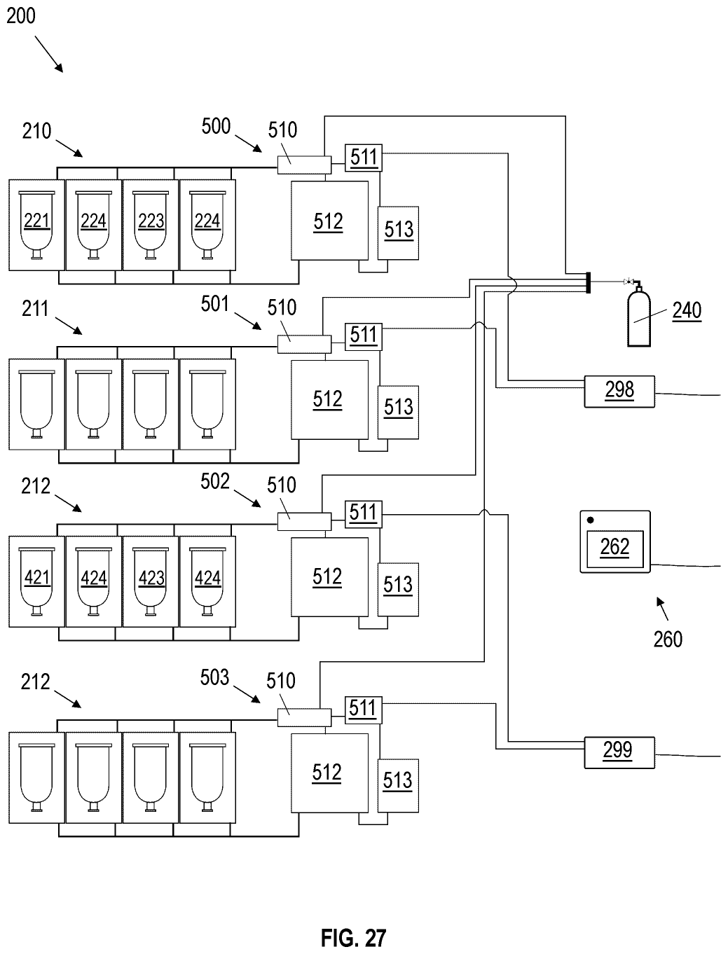

Seaweed system 200 may comprise any number and/or type of seaweed bioreactor systems 210, 211, 212, 213, or the like, including any number or type of seaweed bioreactors like bioreactors 220, 420, or the like, comprising any number of seaweed bioreactor apparatus like apparatus 221, 421, or the like. One example of system 200 is shown in FIG. 26, for example, in which seaweed bioreactor system 210 may consist of seaweed bioreactor apparatus 221, each seaweed bioreactor system 211, 212, and 213 may comprise three (3) seaweed bioreactor apparatus like seaweed bioreactor apparatus 221 or 421, and system controller 260 may be operable with a temperature AC distribution box 298 and life support systems 500, 501, 502, and 503 to control systems 210, 211, 212, and 213 in keeping with any examples described herein. Another example of system 200 is shown in FIG. 27, for example, in which top-level seaweed bioreactor systems 210, 211 are operable with a first temperature AC distribution box 298, life support systems 500, 501, and system controller 260 (e.g., via a wireless connection); and bottom-level seaweed bioreactor systems 212, 213 are operable with a second temperature AC distribution box 299, life support systems 500, 501, and system controller 260 (e.g., via the wireless connection).

As shown in FIGS. 26 and 27, CO₂ and/or air supply 240 is depicted as a single tank buy may comprise any aspects of supplies 140, 240, and/or 340 described herein.

As noted above, each individual seaweed system 210, 211, 212, and 213 may comprise its own life support system 500, 501, 502, and 503, each of which may be independently operable with system controller 260 to maintain optimal growing conditions. Each life support system 500, 501, 502, and 503 may comprise self-contained, independently operable control module operable with little or no support from system controller 260 in certain situations. Exemplary aspects of systems 500, 501, 502, and 503 are now described with reference to system 500. As shown in FIG. 15, for example, system 500 may comprise a CO₂ and/or air supply 510, an AC distribution system 511, a control box 512, a DC power supply 513, a data transceiver 514, and a plurality of cables, tubes, and/or wires extending between.

Aspects of CO₂ and/or air supply 510 may be like aspects of CO₂ and/or air supply 240 or any counterpart thereof described herein. As shown in FIG. 15, for example, CO₂ and/or air supply 510 may comprise CO₂ solenoids and air pumps operable with one or more CO₂ tanks 515 to distribute CO₂ and/or air to each bioreactor apparatus 221, 222, 223, and 224 though a tubing system 516 extending therebetween. AC distribution system 511 may comprise circuitry operable to receive AC power from the grid and direct it to the CO₂ solenoids and air pumps of CO₂ and/or air supply 510 responsive to control signals output from control box 512, making it possible to independently adjust the amount of CO₂ and/or air delivered to each bioreactor apparatus 221, 222, 223, and 224 through tubing system 516. As shown in FIGS. 26 and 27, for example, AC distribution system 511 also may comprise AC distribution boxes 298, 299. DC power supply 513 may comprise a rectifier operable to receive AC power from the circuitry of AC distribution system 511 and convert into DC power for use by control box 512.

As shown in FIG. 15, for example, control box 512 may comprise hardware and/or software operable with DC power from DC power supply 513 to receive data and/or control signals from system controller 260 via data transceiver 514, receive data from a sensor of each bioreactor apparatus 221, 222, 223, and 224 (e.g., a pH or temperature sensor) though a data transceiver 517 extending therebetween, and output control signals to one or both of CO₂ and/or air supply 510 and/or an operating element of any bioreactor apparatus 221, 222, 223, and 224 responsive the received control signals and/or data. Data transceiver 514 and data transceiver 517 are shown as wired data connections but also could be wireless data connections made with BlueTooth, WiFi, or the like.

As described herein and shown in FIGS. 14 and 15, for example, control box 512 may thus be operable to: receive system level control signals from system controller 260 (e.g., sustain or shutdown); receive feedback data from bioreactor apparatus 221, 222, 223, and 224 (e.g., from onboard sensors); concurrently analyze the feedback data for each apparatus 221, 222, 223, and 224 in view of the control signals; generate individualized control signals for one or both of CO₂ and/or air supply 510 and/or an operating element of each apparatus 221, 222, 223, and 224 based its analysis of their respective feedback data; and/or output the individualized control signals to CO₂ and/or air supply 510 and/or the operating element of apparatus 221, 222, 223, and 224.

Like system controller 160 of seaweed system 10 described above, system controller 260 of seaweed system 200 may comprise hardware and/or software operable to maintain optimal conditions for growing seaweed in bioreactor 220 of seaweed bioreactor system 210 as well as each counterpart bioreactor (e.g., like bioreactor 420) of each counterpart seaweed bioreactor system 211, 212, and 213. As shown in FIG. 14, for example, system controller 260 may comprise a housing 261 and a display element 262 operable with one or more processing elements, a data transceiver, and like components.

As shown in FIG. 14, for example, housing 261 may comprise a free-standing structure made of a rigid material. The rigid material may comprise a metallic material (e.g., diamond plate) operable to support and/or contain the elements of system controller 260 on support structure 205 and act as a heat sink for those elements. Processing element 263 may be located in housing 261 and comprise data processing elements operable to receive data from and send control signals to other elements of system 200. For example, processing element 263 may comprise one or more of a processor(s), a memory, and/or a transceiver operable with software (e.g., stored on the memory) to receive and process the data and/or generate and output the control signals described herein.

Display element 262 may comprise a touchscreen display operable with processing element 263 to present a graphical user interface to a user and receive touch-based inputs from that user. As shown in FIG. 14, for example, processing element 263 may comprise a communication bus or any other wired and/or wireless data communication hardware for: establishing and maintaining data connection(s) operable to: receive operating instructions (e.g., from a human and/or computer operator); receive feedback data from life support systems 500, 501, 502, and/or 503; analyze the instructions and/or feedback data with any combination of local (e.g., an onboard chip) and/or remote processing elements (e.g., such as Al-powered system accessible over the internet); generate individualized control signals for each life support system 500, 501, 502, and/or 503 based its analysis of their respective instructions and feedback data; and/or output the individualized control signals to system 500, 501, 502, and/or 503.

Although shown as being proximate to life support systems 500, 501, 502, and 503 in FIG. 14, for example, all or a portion of system controller 260 may be located remotely from seaweed system 200. For example, each control box 512 of life support systems 500, 501, 502, and 503 may comprise a microcontroller (e.g., like microcontroller 40) operable to process data and/or generate control signals locally, meaning that all or a portion of system controller 260 may be in the cloud and operable to receive the data and/or output the control signals over the internet. In keeping with this example, a cloud-based version of system controller 260 (e.g., like system controller 160) may monitor and control any number of life support systems 500, 501, 502, 503, etc. As shown in FIG. 27, for example, a cloud-based embodiment of system controller 260 may be similarly operable with life support systems 500, 501, 502, and 503.

### Examples of a Seaweed Bioreactor Apparatus for Gametophytes

Aspects of this disclosure are now described with reference to exemplary seaweed bioreactor apparatus operable to maintain optimal conditions for growing gametophytes in a volume of culture medium 1. Some aspects are described with reference to seaweed bioreactor apparatus 221 of seaweed system 210 shown in FIG. 14, for example, but could alternatively be described with reference to any bioreactor apparatus 222, 223, or 224 of system 210 and/or any counterpart bioreactor apparatus of seaweed systems 211, 212, or 213. Numerous potential configurations of seaweed bioreactor apparatus 221 are described herein, each having aspects that may be like those of other seaweed bioreactor apparatus described herein but for the differences noted below.

Seaweed bioreactor apparatus 221 may be operable with life support system 500 and system controller 260 to contain and grow a culture of seaweed cells in a volume of culture medium 1. Aspects of bioreactor apparatus 221 may be optimized to maintain optimal conditions for growing gametophytes in the volume of culture medium 1. As shown in FIGS. 14, 15, 16, and/or 17, for example, seaweed bioreactor apparatus 221 may comprise an enclosure 230, a housing 231, a mounting assembly 232, a motor 233, a temperature control unit 234, a culture medium sensor 235, a light source 336, a vent filter 337, a port 238, a port 239, and a valve 300.

As shown in FIGS. 14 and 16, for example, enclosure 230 may be made of a rigid material that seals off housing 231 from ambient light, making it possible to carefully control the light spectrum applied to any seaweed cells that are contained in a volume of culture medium 1 sealed in housing 231. The rigid material may comprise wood and/or a polymeric material like wood. As shown in FIG. 16, for example, enclosure 230 may comprise exterior walls 244, an access door 245, a light-filtering window 246, and a flow control meter 364.

Like enclosure 30, enclosure 230 also may be light-tight. Because it may include light-filtering window 246, however, enclosure 230 (e.g., like enclosure 30) also may be described as light-controlling, meaning that it may be light-tight or operable to selectively admit a filtered portion of ambient light into enclosure 230. As shown in FIG. 16, for example, exterior walls 244 of enclosure 230 may comprise six (6) walls attached at their ends to define a rectangular shape. A front wall 244 may comprise an access opening and access door 245 moveably attached thereto (e.g., with hinges), making it easy to access the interior of enclosure 230 through front wall 244. Light-filtering window 246 may span over an opening extending through access door 245 and be attached thereto. As shown in FIG. 16, for example, enclosure 230 may be a light-tight enclosure (e.g., like enclosure 30 described above) save for light-filtering window 246 that may be always open or selectively opened (e.g., by removing a cover or opening a shade) to introduce a particular wavelength of light to the seaweed contained in enclosure 230. In this example, light-filtering window 246 may comprise a blue light filtering element operable to permit viewing inside enclosure 230 without changing the seaweed culture to “sexual” by exposing it to blue light, for example, in which changing to sexual means inducing the seaweed biomass to form eggs and gametes responsive to the blue light (or another hue).

To avoid contamination, seaweed bioreactor apparatus 221 may be operated for extended periods of time (e.g., weeks or months) without opening access door 245. Light-filtering window 246 may permit viewing inside enclosure 230 and always or selectively exposing the seaweed culture to a particular light during that time. Flow control meter 364 may output temperature data associated with a temperature outside enclosure 230 to life support system 500 at regular intervals during that time.

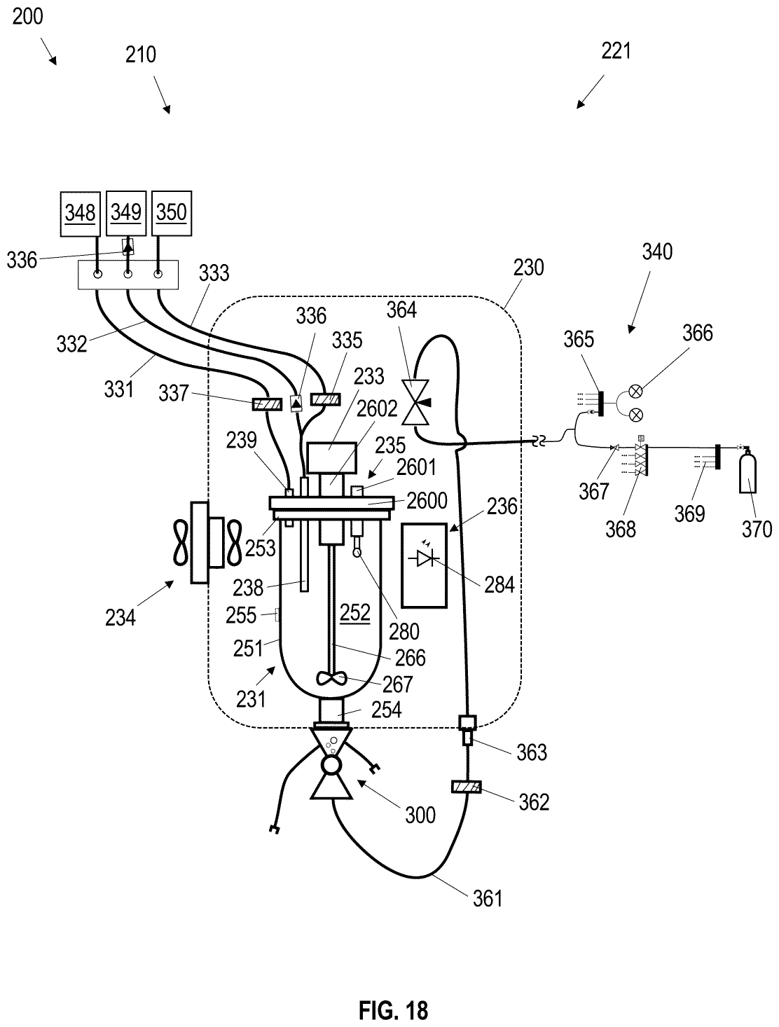

As shown in FIG. 17, for example, housing 231 may comprise a wall 251 defining a vessel 252 sized to contain the volume of culture medium 1. As shown in FIG. 18, for example, vessel 252 may comprise a cylindrical shape with an open top having a flange 253 and an open bottom having a flange 254. In contrast to housing 31 described above, housing 231 may not include a thermal jacket, instead relying upon temperature control unit 234 to reduce its weight and the number of fluidic connections in system 200. Because it is lighter, housing 231 may be suspended from interior surfaces of enclosure 230 with undersurfaces of flange 253.

As shown at right in FIG. 17, for example, mounting assembly 232 may comprise a mounting plate 260, a sensor support tube 261, a bearing shaft 262, a motor mount 263, a drive shaft 266, and a blade 267. Like other mounting assemblies described herein, mounting assembly 232 also may be autoclavable.

As shown in FIG. 17, for example, mounting plate 260 may be removably attached to flange 253 with a rim seal 270 operable to seal vessel 252 and prevent ambient air from entering vessel 252. Sensor support tube 261 may comprise a hollow tube that is attached to and extends through mounting plate 260 toward the open bottom of vessel 252. Bearing shaft 262 may comprise a hollow tube that is attached to mounting plate 260 and extends upwardly therefrom. Motor mount 263 may be fixedly attached to a top portion of bearing shaft 262 and comprise an opening extending therethrough. A bottom portion of bearing shaft may extend through mounting plate 260.

Motor 233 may comprise any type of electric motor operable with control signals output from control box 512 of life support system 500. As shown at right in FIG. 17, for example, motor 233 may be attached to the top portion of motor mount 263 to form an airtight seal that prevents ambient air from entering vessel 252. An output shaft of motor 233 may be directed into vessel 252 through the opening of motor mount 263 and the hollow tube of bearing shaft 262. In keeping with above, one end of drive shaft 266 may be attached to the output shaft of motor 233 with a rotational bearing operable to further prevent ambient air from entering vessel 52 (e.g., like drive shaft coupler 65) and the other end of drive shaft 266 may be attached to blade 267, which itself may be like any blade 67, 67-1, 67-2, 67-3, 67-4, 67-5, 67-6, or 67-7 described herein.

Temperature control unit 234 may comprise any cooling device operable with control signals output from control box 512 of life support system 500. As shown in FIG. 18, for example, temperature control unit 234 may comprise a solid-state air conditioning device that utilizes fans and Peltier coolers to cool vessel 252 responsive to a temperature sensor 255 attached to wall 251 of housing 231, flow control meter 364 attached to enclosure 230, control signals from control box 512 of life support system 500 (e.g., as shown in FIG. 15), and/or control signals from system controller 260. A mounting surface of temperature control unit 234 may be attached to one wall 266 of enclosure 230, a cooling portion of temperature control unit 234 may comprise the Peltier coolers and a fan extending through an opening of enclosure 30 to position them closer to housing 31. By way of example, temperature control unit 234 may comprise an ACT-TEC Thermoelectric Air Conditioner like those sold by Advanced Cooling Technologies or any of their competitors.

As shown in FIG. 18, for example, culture medium sensor 235 may output data associated with one or more characteristics of the volume of culture medium 1 in vessel 252 to life support system 500 (e.g., as shown in FIG. 15). Aspects of culture medium sensor 235 may be like aspects of culture medium sensor 35 described above. As shown in FIG. 18, for example, culture medium sensor 235 may be mounted in sensor support tube 261 to prevent ambient air from entering vessel 252 and position a sensing end 280 of sensor 235 in vessel 252. As above, sensing end 280 may comprise a pH sensor operable to output data associated with a pH level of culture medium 1 using a power/data connection, such as a 4 pin, military-style analog connection.

Light source 236 may comprise any lighting device operable with control signals output from control box 512 of life support system 500 (e.g., as shown in FIG. 15). Aspects of light source 236 may be like aspects of light source 36 described above. As shown in FIG. 18, for example, light source 236 may be similarly mounted on an interior surface of enclosure 230 and comprise different clusters and/or strips of LEDs that are directed towards translucent enclosure 231 and operable with life support system 500 to direct different hues of light toward housing 231, such as blue hues, red hues, and any other hues appropriate for growing seaweed.

Port 238 may comprise piping and/or tubing operable to remove effluent from the volume of culture medium 1 in vessel 252 and add additional volumes of culture medium 1 to vessel 252. A shown in FIG. 18, for example, one end of port 238 may comprise a delivery tube extending through mounting plate 260 into an interior portion of vessel 252 and another end of port 238 may comprise a Y-connector with a first branch attached to a tube 332 in fluid communication with a supernatant outlet 349 and a second branch attached to a tube 333 in fluid communication with a culture medium supply 350. Supernatant outlet 349 may comprise a syringe port operable to manually remove amounts of supernatant from the volume of culture medium 1 and/or a pump operable to remove the amounts of supernatant responsive to life support system 500. As shown in FIG. 18, for example, tube 332 may comprise a check valve 336 operable to prevent the supernatant from flowing back into vessel 252.

Aspects of culture medium supply 350 may be like aspects of culture medium supply 150 described above. For example, culture medium supply 350 may comprise a pump or solenoid operable to add the additional volumes of culture medium 1 responsive to control signals from life support system 500. As shown in FIG. 18, for example, line 333 may comprise a media filter 335 operable to prevent contaminants from entering vessel 252, such as a mechanical water filter and/or an in-line UV-C LED sterilizer operable with control signals from life support system 500 to sterilize the added culture medium 1 before it enters vessel 252.

Port 239 may comprise piping and/or tubing operable to remove exhaust gasses from vessel 252, such as excess amounts of CO₂ and/or air that might otherwise over-pressurize the head space above culture medium 1. As shown in FIG. 18, a bottom end of port 239 may extend into the head space and a top end of port 239 may be attached to a tube 331 in fluid communication with a vent exhaust 348, providing a pathway for removing the excess amounts of CO₂ and/or air from the head space. Vent filter 337 may prevent unfiltered ambient air from entering vessel 252 if a vacuum is formed therein by any of the operating methods described. As shown in FIG. 18, for example, vent filter 337 may be attached to the top end of port 239 to prevent ambient air from entering vessel 252.

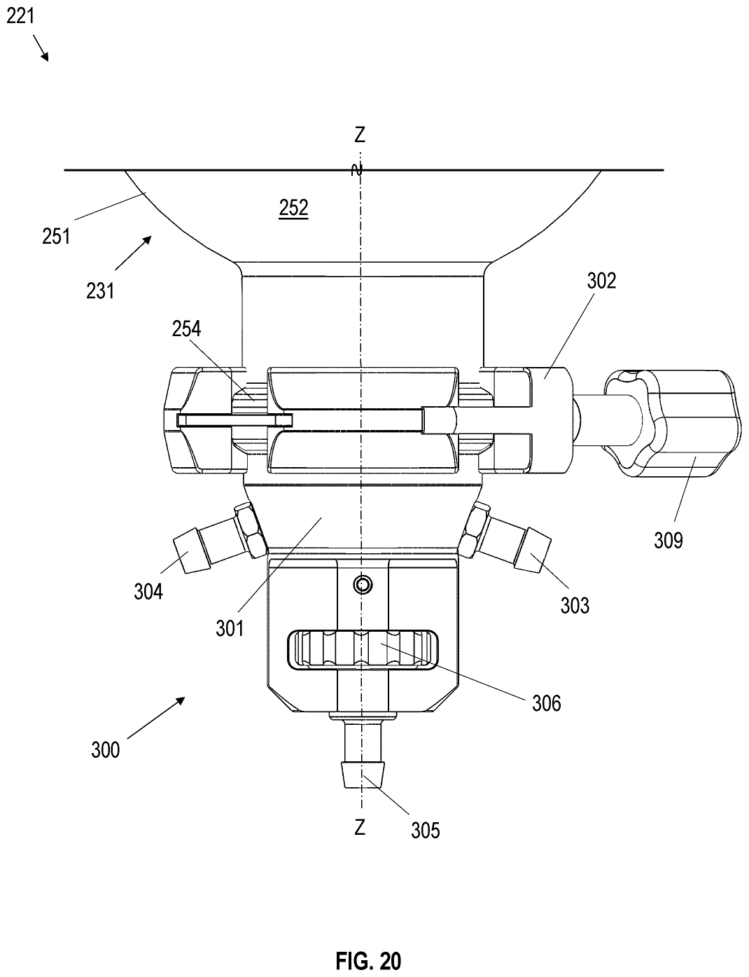

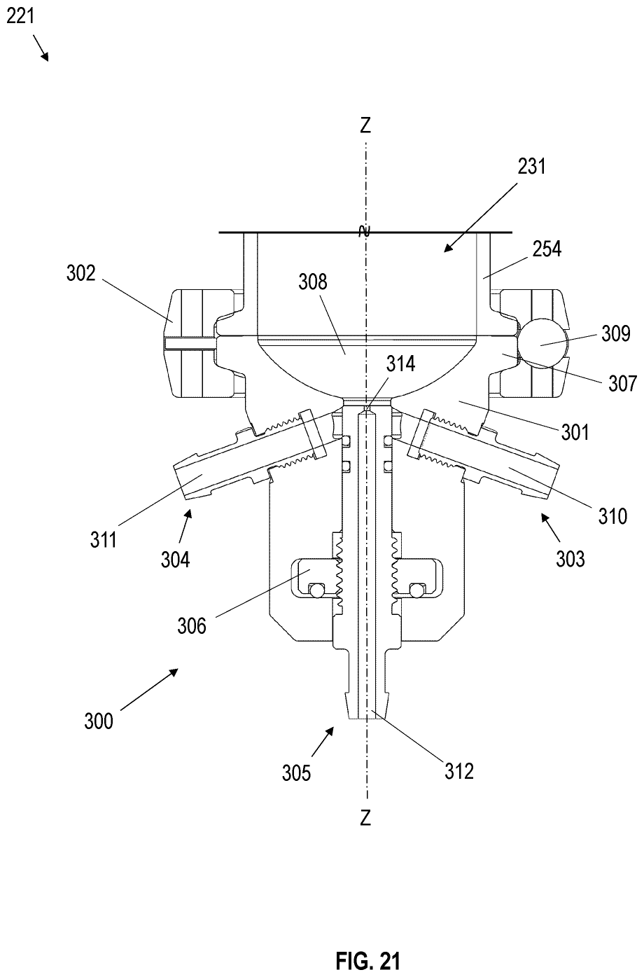

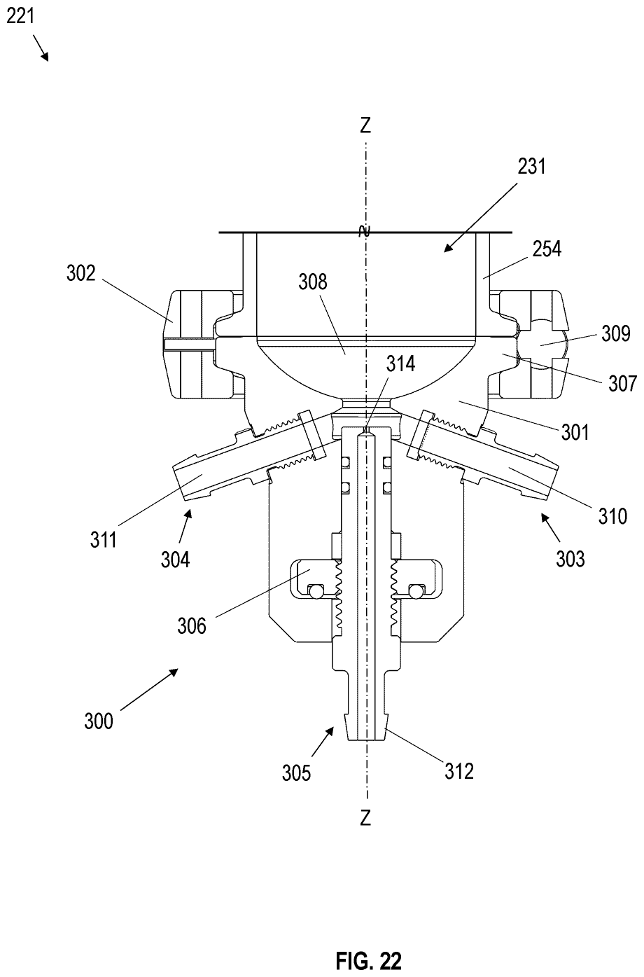

As shown in FIG. 17, for example, valve 300 may seal the open bottom of vessel 252, diffuse CO₂ and/or air into culture medium 1 in vessel 252 and remove culture medium 1 from vessel 252. As shown in FIG. 22, for example, valve 300 also may be operable in a cleaning mode that prevents contaminants from entering vessel 252. For at least these reasons, valve 300 of seaweed bioreactor apparatus 221 may possess certain advantages over port 38 of bioreactor apparatus 21, even though either valve 300 or port 38 may be effectively used to diffuse CO₂ and/or air into to culture medium 1. As shown in FIGS. 20, 21, and/or 22, for example, valve 300 may comprise a valve body 301, a rim seal 302, a removal port 303, a cleaning port 304, a delivery port 305, and a control wheel 306.

As shown in FIGS. 20 and 21, for example, valve body 301 may comprise a generally cylindrical rigid structure formed (e.g., 3D printed or machined from metal) to define a top portion with a flange 307 and a drainage chamber 308. Flange 307 may be attached to flange 254 of translucent enclosure 231 with rim seal 302 to seal the open bottom of vessel 252 and drainage chamber 308. Rim seal 302 (e.g., like rim seal 270) may comprise a threaded actuator 309 operable to prevent leaks by applying clamping pressure to flanges 254, 307. Removal port 303 may be threaded into a first channel of valve body 301 and have interior surfaces defining a removal port channel 310 leading to drainage chamber 308. Cleaning port 304 may be threaded into a second channel of valve body 301 and have interior surfaces defining a cleaning port channel 311 leading to drainage chamber 308.

As shown in FIG. 21, for example, delivery port 305 may be slidably mounted inside a third channel of valve body 301 and have interior surfaces defining a delivery port channel 312 leading to drainage chamber 308. A bottom portion of delivery port 305 and delivery port channel 312 may receive CO₂ and/or air (e.g., from CO₂ and/or air supply 340 described below) and direct it towards drainage chamber 308. A top portion of delivery port 305 may comprise a nozzle 314 operable to diffuse the received CO₂ and/or air into drainage chamber 308, allowing it to bubble upwards through culture medium 1.

As shown in FIG. 21, for example, control wheel 306 may be rotatably attached to valve body 301 and comprise a central opening extending therethrough. Delivery port 305 may be movable through the third channel of valve body 301 and the central opening of control wheel 306. Delivery port 305 may comprise exterior threads engageable with corresponding interior threads of control wheel 306 so that rotating it in a first direction (e.g., clockwise) moves delivery port 305 toward drainage chamber 308 and rotating wheel 306 in a second direction (e.g., counterclockwise) moves delivery port 305 away from chamber 308. As shown in FIG. 21, for example, delivery port 305 may close removal port 303 and cleaning port 304 when control wheel 306 is rotated in the first direction until exterior surfaces of port 305 are positioned in front of ports 303 and 304, sealing them off from chamber 308. Conversely, as shown in FIG. 22, for example, delivery port 305 may open removal port 303 and cleaning port 304 when control wheel 306 is rotated in the second direction until exterior surfaces of port 305 are positioned away from ports 303 and 304 to establish a fluid communication with chamber 308. Because nozzle 314 is on a top surface of delivery port 305, the flow of nozzle 314 will not be affected by the opening and/or closing of ports 303 and 304.

CO₂ and/or air supply 340 may comprise electromechanical devices operable with control signals output from control box 512 of life support system 500. Exemplary aspects of CO₂ and/or air supply 340 are now described with reference to a configuration of electromechanical devices optimized for use with seaweed bioreactor 220 of seaweed system 210, which is shown for example in FIG. 14 as including four independently operable bioreactor apparatus 221, 222, 223, and 224. The described aspects are scalable, meaning that some aspects may be repeated for use with counterpart seaweed bioreactors in seaweed systems 211, 212, 213, and the like. As shown in FIG. 18, for example, CO₂ and/or air supply 340 may comprise a tube 361, an inlet filter 362, an adapter 363, a flow control meter 364, an air pump 365, a flow throttle 367, a CO₂ solenoid bank 368, a CO₂ splitter 369, and a CO₂ tank 370.

As shown in FIG. 18, for example, tube 361 may extend from delivery port 305 toward a Y-connector having a first branch leading toward air pump 365 and a second branch leading toward CO₂ tank 370. Each of inlet filter 362, adapter 363, and flow control meter 364 may be in-line with tube 361 at locations prior to the Y-connector. Inlet filter 362 may remove contaminants from the CO₂ and/or air before it enters delivery port 305 and is diffused into culture medium 1. Adapter 363 may extend through an opening of enclosure 30 and be attached thereto, allowing tube 361 to pass into enclosure 30 without admitting ambient air. Flow control meter 364 may be attached to an interior surface of enclosure 30 and operable to output data associated with a volumetric flow rate of CO₂ and/or air flowing through tube 361 toward vessel 35 to life support system 500.

Air pump 365 may comprise one or more electric air pumps operable with control signals output from control box 512 of life support system 500. As shown in FIG. 18, for example, air pump 365 may comprise a plurality of air pumps operable responsive to the control signals and a distribution panel including individual outputs for seaweed bioreactor apparatus 221, 222, 223, and 224 of seaweed bioreactor 220. The first branch of tube 361 after the Y-connector may comprise a check valve operable to prevent CO₂ from leaking out of air pump 365 when not activated by the control signals. The second branch of tube 361 after the Y-connector may comprise flow throttle 367, CO₂ solenoid bank 368, CO₂ splitter 369, and CO₂ tank 370. CO₂ splitter 369 may include individual outputs for seaweed bioreactor apparatus 221, 222, 223, and 224. CO₂ solenoid bank 368 may be operable with control signals from life support system 500 to direct one or more flows of CO₂ from CO₂ tank 370 into the individual outputs. Flow throttle 367 may affect the ratio of CO₂ to air flowing from the one or more outputs.

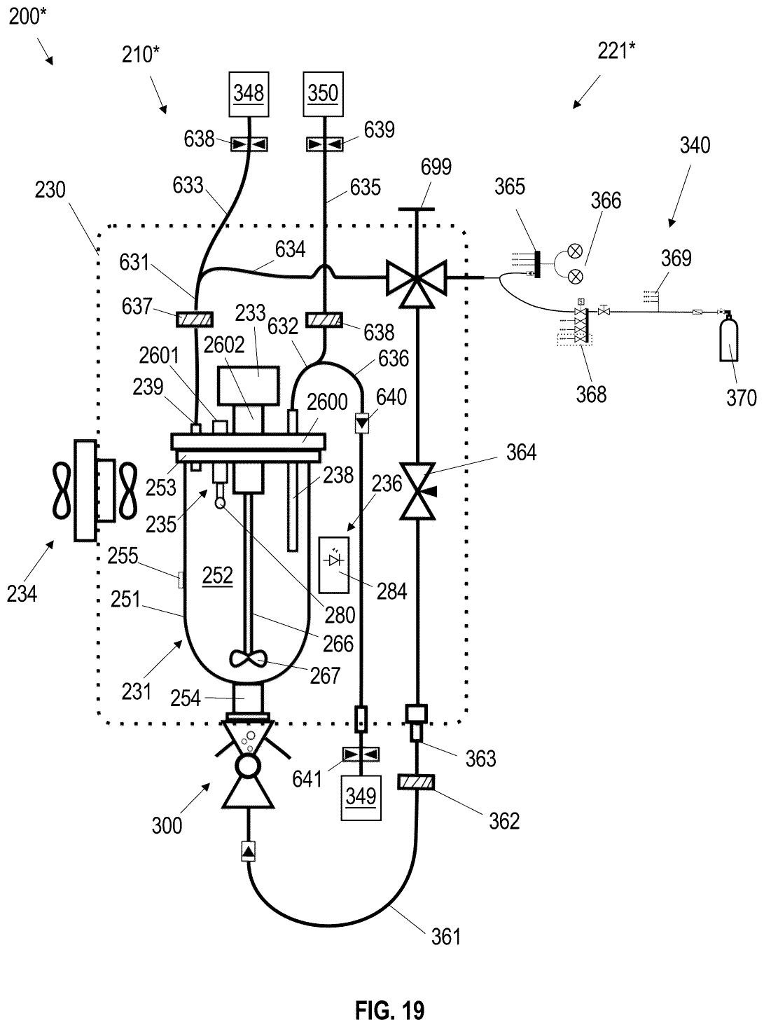

As described above, seaweed bioreactor system 210 may utilize negative pressure to remove the supernatant and/or effluents from the volume of culture medium 1, such as by removing them manually with a syringe or automatically with a pump. An alternative seaweed bioreactor system 210* is shown in FIG. 19, for example, as a modified embodiment of system 210 with an alternate tubing configuration operable to remove the supernatant and/or effluent from vessel 252 in an even more biosecure manner. In contrast to system 210, for example, seaweed bioreactor system 210* may utilize positive pressure to remove the supernatant and/or effluent without opening vessel 252 and/or otherwise exposing culture medium 1 to ambient air or other contaminants, as may occur when using a syringe.

As shown in FIG. 19, for example, port 239 may be attached to a tube 631 comprising a Y-connector with a first branch 633 leading to vent exhaust 348 and a second branch 634 leading to a first port of a three-way valve 699 (e.g., facing left). Tube 631 may comprise an in-line filter 637 such as a vent and blanket air filter. First branch 633 may comprise a valve 638 operable to stop flows to and from vent exhaust 348. As shown in FIG. 19, for example, port 238 may be attached to a tube 632 comprising a Y-connector with a first branch 635 leading to culture medium supply 350 and a second branch 636 leading to supernatant outlet 349. First branch 635 may comprise a valve 639 operable to stop flows to and from culture medium supply 350. Second branch 636 may comprise a check valve 640 operable to prevent flows of supernatant and/or effluent back into vessel 252 from supernatant outlet 349 should it back up. As shown in FIG. 19, for example, supernatant outlet 349 may comprise a basin or drain that receives amounts of supernatant and/or effluent removed from culture medium 1.

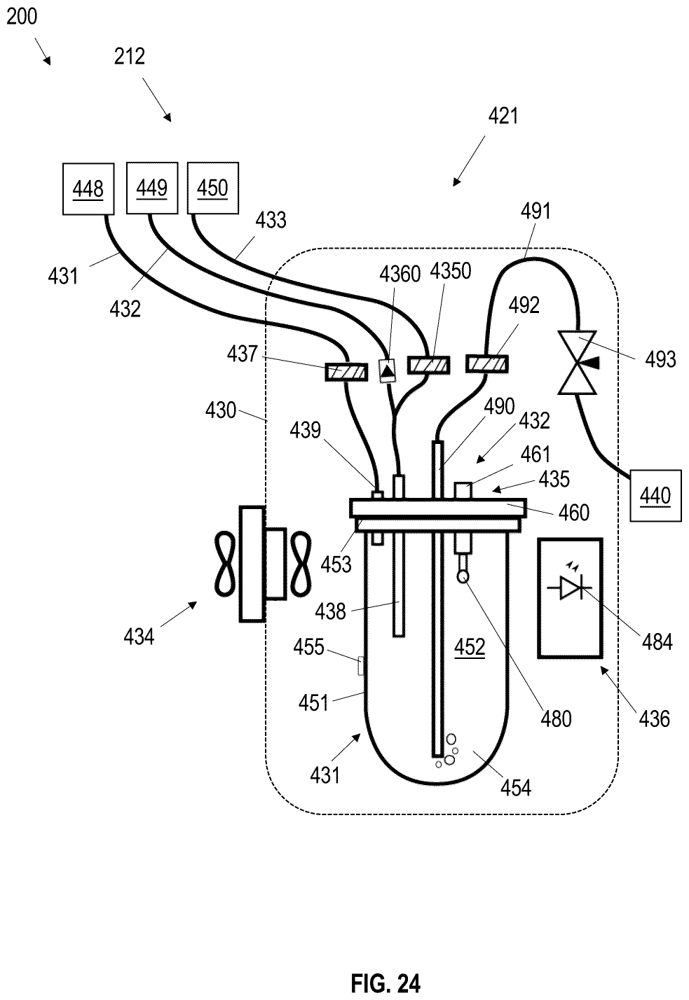

As shown in FIG. 19, for example, each of CO₂ and/or air supply 340, tube 361, inlet filter 362, adapter 363, flow control meter 364, air pump 365, flow throttle 367, CO₂ solenoid bank 368, CO₂ splitter 369, and CO₂ tank 370 may be like as described above with reference to FIG. 24. In seaweed system 200*, however, tube 361 may lead to a second port of a three-way valve 699 (e.g., facing down) and the CO₂ and/or air output from supply 340 may be output to a third port of a three-way valve 699 (e.g., facing right).

Because of its tubing configuration, seaweed bioreactor system 210* may thus be operable to divert the CO₂ and/or air output from supply 340 into the head space above culture medium 1 by causing supply 340 to output a flow of CO₂ and/or air, opening the first port of three-way valve 699 and closing pinch valve 638 so that the flow of CO₂ and/or air enters the head space of vessel 252 and creates a positive pressure. If more and/or faster pressurization is needed, then the second port of three-way valve 699 also may be closed to ensure that all of CO₂ and/or air output from supply 340 is utilized.

Once the positive pressure has been obtained, seaweed bioreactor system 210* may then be operable to remove supernatant and/or effluent from culture medium 1 without opening vessel 252 and/or otherwise exposing culture medium 1 to ambient air. As shown in FIG. 19, for example, closing valve 639 and opening valve 641 after positively pressurizing the head space will cause a flow of supernatant and/or effluent to flow out of port 238, through second branch 363 of tube 632, and into supernatant outlet 649 for disposal. Port 238 may only extend toward an upper or middle portion of housing 231, where most of the supernatant and/or effluent may be found, which may reduce the amount of positive pressure required for their removal. As shown in FIG. 19, for example, the Y-connector of line 632 may be angled to prevent additional volumes of culture medium 1 from source 150 from flowing into outlet 649 when system 210* is not removing supernatant and/or effluent from vessel 252.

As shown in FIG. 19, for example, each valve in seaweed bioreactor system 210* may be electronically actuated with control signals from life support system 500 so that system 210* may be switched into a supernatant and/or effluent removal mode based on a predetermined schedule and/or responsive data associated with culture medium 1, such as that output by culture medium sensor 235 (e.g., pH level) or another sensor. Of course, any valve in seaweed bioreactor system 210* also may be manually operated. For example, each of valves 638, 639, and 641 may comprise pinch valves.

### Examples of a Seaweed Bioreactor Apparatus for Sporophytes

Aspects of this disclosure are now described with reference to exemplary seaweed bioreactor apparatus operable to maintain optimal conditions for growing sporophytes in a volume of culture medium 1. Some aspects are described with reference to seaweed bioreactor apparatus 421 of seaweed system 212 shown in FIG. 14, for example, but could alternatively be described with reference to any bioreactor apparatus 422, 423, or 424 of system 212 and/or any counterpart bioreactor apparatus of seaweed systems 210, 211, or 213. Numerous potential configurations of seaweed bioreactor apparatus 421 are described herein, each having aspects that may be like those of other seaweed bioreactor apparatus described herein but for the differences noted below.

Seaweed bioreactor apparatus 421 may be operable with life support system 502 and system controller 260 to contain and grow a culture of seaweed cells in a volume of culture medium 1. Aspects of bioreactor apparatus 421 may be optimized to maintain optimal conditions for growing sporophytes in culture medium 1. As shown in FIGS. 23, 24, and/or 25, for example, seaweed bioreactor apparatus 421 may comprise an enclosure 430, a housing 431, a mounting assembly 432, a temperature control unit 434, a culture medium sensor 435, a light source 436, a port 438, a port 439, and a straw 490.

Aspects of enclosure 430 may be like their counterpart aspects of enclosure 230 described above but within the 400 series of numbers, such as exterior walls 444, access door 445, light-filtering window 446, and ambient temperature sensor 447. In this example, light-filtering window 446 may comprise a light filtering element operable to change the seaweed culture to “sexual” by admitting blue light into enclosure 30. In keeping with above, enclosure 430 also may be described as light-tight or light-controlling according to the examples provided herein.

To avoid contamination, seaweed bioreactor apparatus 421, like apparatus 221, also may be operated for extended periods of time (e.g., weeks or months) without opening access door 445. Light-filtering window 446 may permit viewing inside enclosure 430 and selectively affecting the seaweed culture during that time. Ambient temperature sensor 447 may output temperature data associated with the temperature outside enclosure 430 to life support system 502 at regular intervals during that time.

As shown in FIG. 23, for example, housing 431 may comprise a wall 451 defining a vessel 452 sized to contain the volume of culture medium 1. As shown in FIG. 24, for example, vessel 452 may comprise a cylindrical shape with an open top having a flange 453 and closed bottom 454. In contrast to housing 31 described above, housing 431, like housing 231, also may not include a thermal jacket, instead relying upon temperature control unit 435. Housing 431 may be suspended from interior surfaces of enclosure 430 with undersurfaces of flange 453. In keeping with above, vessel 452 also may be described as a light-transmitting vessel.

By omitting motor 233, blade 267, and associated elements, mounting assembly 432 may be comparatively simpler than mounting assembly 232. As shown at right in FIG. 24, for example, mounting assembly 432 may comprise a mounting plate 460, a sensor support tube 461, and a support rack 462. Like other mounting assemblies described herein, mounting assembly 232 also may be autoclavable.

As shown in FIG. 23, for example, mounting plate 460 may be removably attached to flange 453 with a threaded connection operable to seal vessel 252 and prevent ambient air from entering vessel 452. Sensor support tube 461 may comprise a hollow tube that is attached to and extends through mounting plate 460 toward the closed bottom of vessel 452. Support structure 262 may comprise rigid tubes and plates that are attached to mounting plate 260 and extend upwardly therefrom to provide vertical supports for port 438, port 439, and straw 490.

Aspects of temperature control unit 434, temperature sensor 435, culture medium sensor 435, light source 436, port 438, and port 439 shown in FIG. 24, for example, may be identical to aspects of temperature control unit 234, temperature sensor 235, culture medium sensor 235, light source 236, port 238, and port 239 described above and shown in FIG. 18, for example, except that elements of system 412 may output data to and/or be responsive to control signals from life support system 502 rather than life support system 500 of system 410.

A shown in FIG. 24, for example, one end of port 438 may comprise a delivery tube extending through mounting plate 460 into an interior portion of vessel 452 and another end of port 438 may comprise a Y-connector with a first branch attached to a tube 432 in fluid communication with an effluent withdraw 349 and a second branch attached to a tube 433 in fluid communication with a culture medium supply 450. Effluent withdraw 449 may lead to drain or other form of waste disposal. Like supernatant outlet 349, effluent withdraw 449 also may comprise a syringe port operable to manually remove amounts of supernatant from the volume of culture medium 1 and/or a pump operable to remove the amounts of effluent responsive to life support system 502. As shown in FIG. 24, for example, tube 432 also may comprise a check valve 436 operable to prevent the supernatant from flowing back into vessel 452.

Aspects of culture medium supply 450 may be identical to counterpart aspects of culture medium supplies 350 described above, including line 433 and media filter 435. Aspects of port 439 such as vent exhaust 448 and vent filter 437 also may be identical to counterpart aspects of port 439 described above. Because diffusing valve 300 is omitted from seaweed bioreactor apparatus 421 (e.g., FIG. 24), straw 490 may utilized to deliver CO₂ and/or air to a bottom portion of vessel 452 in a manner like port 38 of seaweed bioreactor apparatus 21 (e.g., FIG. 4). A shown in FIG. 24, for example, a bottom end of straw 490 may extend into in the bottom portion of vessel 452 and a top end of straw 490 may be attached to a tube 491 in fluid communication with CO₂ and/or air supply 440. An inlet filter 492 and a flow control meter 493 may be in-line with tube 491 at locations prior to CO₂ and/or air supply 440. Inlet filter 392 may remove contaminants from the CO₂ and/or air before it enters straw 490. Flow control meter 492 may be attached to enclosure 430 and operable to output data associated with a volumetric flow rate of CO₂ and/or air flowing through tube 491 to life support system 502.

As shown in FIG. 24, for example, aspects of CO₂ and/or air supply 440 of seaweed bioreactor system 212 may be identical to counterpart aspects of CO₂ and/or air supply 240 of seaweed bioreactor system 210, but with electromechanical devices operable with control signals output from a counterpart control box (e.g., like control box 512) of life support system 502. Because these aspects are scalable, CO₂ and/or air supplies 240, 440 may be part of the same CO₂ and/or air supply system and thus optimized to deliver CO₂ and/or air supply to any seaweed bioreactor apparatus of seaweed system 210, 211, 212, and 213.

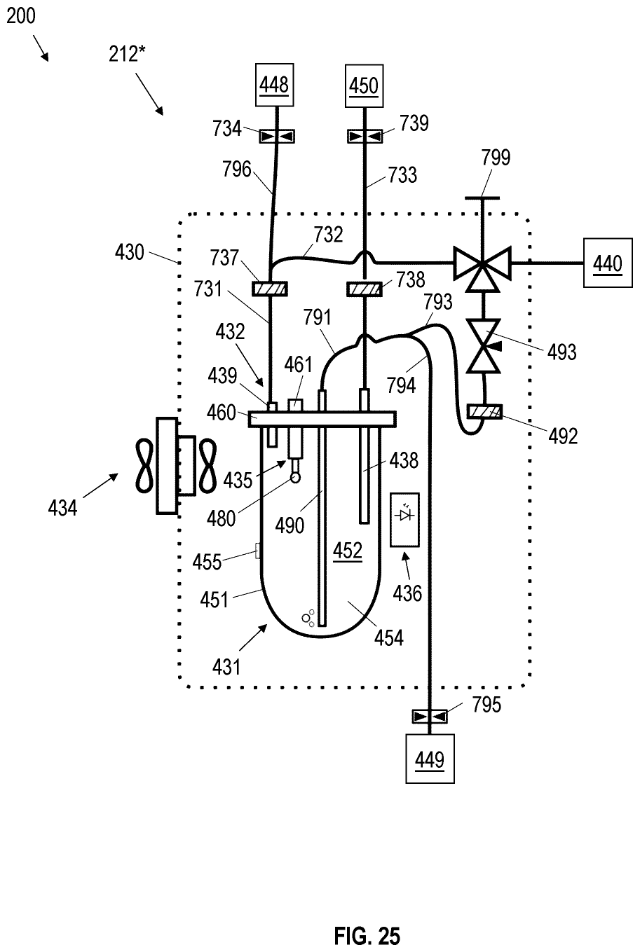

As described above, seaweed bioreactor system 212 may utilize negative pressure to sample or harvest the sporophytes growing in the volume of culture medium 1, such as by removing them manually with a syringe or automatically with a pump. An alternative seaweed bioreactor system 212* is shown in FIG. 25, for example, as a modified embodiment of system 212 with an alternate tubing configuration operable to sample or harvest the sporophytes from vessel 452 in an even more biosecure manner. In contrast to system 212, for example, seaweed bioreactor system 212* may utilize positive pressure to sample or harvest the sporophytes without opening vessel 452 and/or otherwise exposing culture medium 1 to ambient air or other contaminants, as may occur when using a syringe.

As shown in FIG. 25, for example, port 439 may be attached to a tube 731 comprising a Y-connector with a first branch 734 leading to vent exhaust 448 and a second branch 732 leading to a first port of a three-way valve 799 (e.g., facing left). Tube 731 may comprise an in-line filter 737 such as a vent and blanket air filter. First branch 734 may comprise a valve 738 operable to stop flows to and from vent exhaust 448. As shown in FIG. 25, for example, port 438 may be attached to a tube 733 leading to culture medium supply 450 and comprising a valve 639 operable to stop flows to and from supply 450.

As shown in FIG. 25, for example, each of CO₂ and/or air supply 440, vent exhaust 448, culture medium supply 450, inlet filter 492, and flow control meter 493 may be like as described above with reference to FIG. 24. In seaweed bioreactor system 210*, however, straw 490 may attached to a tube 791 comprising a Y-connector with a first branch 793 leading to a second port of a three-way valve 799 (e.g., facing down) and a second branch 794 leading to a withdraw outlet 749. First branch 793 may be in-line with inlet filter 492 and flow control meter 493. Second branch 795 may comprise a valve 795 operable to stop flows to and from withdraw outlet 749. As shown in FIG. 25, for example, withdraw outlet 449 may comprise a tube or container that collects amounts of sporophytes sampled or harvested from culture medium 1; and CO₂ and/or air output from supply 440 may be output to a third port of a three-way valve 699 (e.g., facing right).

Because of its tubing configuration, seaweed bioreactor system 410* may thus be operable to divert the CO₂ and/or air output from supply 440 into the head space above culture medium 1 by causing supply 440 to output a flow of CO₂ and/or air, opening the first port of three-way valve 799, and closing valve 738 so that the flow of CO₂ and/or air enters the head space of vessel 452 and creates a positive pressure.

Once the positive pressure has been obtained, seaweed bioreactor system 410* may then be operable to sample or harvest the sporophytes in culture medium 1 without opening vessel 452 and/or otherwise exposing culture medium 1 to ambient air. As shown in FIG. 25, for example, closing valve 739 and opening valve 795 after positively pressurizing the head space will cause a flow of culture medium 1 containing sporophytes to flow out of straw 490, through second branch 794 of tube 791, and into withdraw outlet 449 for collection. As shown in FIG. 25, for example, straw 490 may extend toward closed bottom 454 of housing 431; whereas port 238 seaweed bioreactor system may only extend toward an upper or middle portion of housing 431, where most of the supernatant and/or effluent may be found.

As shown in FIG. 25, for example, each valve in seaweed bioreactor system 212* may be electronically actuated with control signals from life support system 500 so that system 212* may be switched into a sampling or harvesting mode based on a predetermined schedule and/or responsive data associated with culture medium 1, such as that output by culture medium sensor 435 (e.g., pH level) or another sensor. Of course, any valve in seaweed bioreactor system 212* also may be manually operated. For example, each of valves 738, 739, and 795 may comprise pinch valves.

Exemplary Methods of Assembling Seaweed Bioreactor Apparatus and Systems

Methods of assembling seaweed bioreactor apparatus 21 are now described with continued reference to above. Floor plate 41, rear wall 42, and rear top plate 43 of light-tight enclosure 30 may be attached to one another to form an open rigid structure like the example shown in FIG. 7. As shown in FIGS. 3 and 7, for example, the elements of light source 36 may be attached and thermally coupled to rear wall 42. As shown in FIGS. 8 and/or 9, for example, housing 86 may be mounted to rear wall 42 and the elements of microcontroller 40 located therein. As shown in FIG. 3, for example, pump box 77 of pump 34 may be attached to rear top plate 43 with pump mount 76.

As shown in FIG. 5, for example, mounting plate 60, sensor support tube 61, bearing shaft 62, motor mount 63, filter mount 64, driveshaft coupler 65, drive shaft 66, and blade 67 may be attached to one another to form an autoclavable structure like that shown in FIG. 5, which may then be placed in an autoclave and sterilized. Motor 33 may be attached to motor mount 63 after the autoclavable structure has been sterilized. As shown in FIG. 4, for example, mounting plate 60 may then be attached and sealed onto translucent vessel 31 using a sealing element such as rim seal 70.

As shown in FIGS. 3, 4, 5, and/or 7, for example, translucent vessel 31 may be slid into the open rigid structure shown in FIG. 7 and front top plate 45 may be attached to rear wall 42 and interconnected with rear top plate 43 so that the opening for sensor support tube 61, bearing shaft 62, filter mount 63, and holes 68, 69 (e.g., FIGS. 2 and 3) are located inside of receiving opening 46. The top surfaces of mounting plate 60 will now be maintained against the bottom surfaces of plates 43, 45 to prevent ambient light from reaching housing 31 through the top of seaweed bioreactor apparatus 21. As shown in FIG. 3, for example, an inlet tube 90 may be connected to inlet 55 and an opaque valve assembly 94 extending through rear wall 42; and a tube 93 may be connected to outlet 56 and an opaque valve assembly 94 extending through rear wall 42.

As shown in FIGS. 3 and/or 4, for example, culture medium sensor 35 may be slid into sensor support shaft 61 and removably attached to mounting plate 60 to maintain a position of sensing end 80 inside of vessel 52. Various electrical/data connections may be made to facilitate output sensory data from culture medium sensor 35 and control of seaweed bioreactor apparatus 21 that data and/or other data. As shown in FIG. 8, for example, a power/data cable 95 may place motor 33 in electrical/data communication with microcontroller 40 and/or system controller 160; a power/data cable 96 may place pump 34 in electrical/data communication with microcontroller 40 and/or system controller 160; and a power/data cable 97 may place light source 36 in electrical/data communication with microcontroller 40 and/or system controller 160. As shown in FIG. 7, for example, a power/data cable 95 may similarly place communication end 81 of culture medium sensor 35 in electrical/data communication with microcontroller 40 and/or system controller 160. Any wired and/or wireless connections may be utilized.

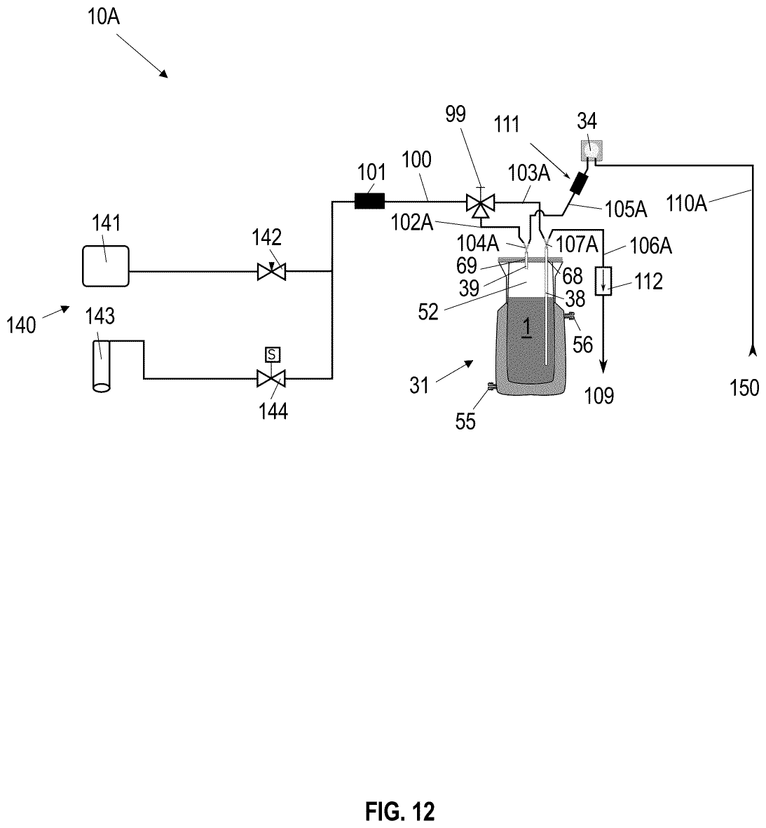

As shown in FIG. 4, for example, vent filter 37 may be attached to filter mount 64 with valve 71. As shown in FIG. 7, for example, a three-way valve 99 may be attached to a side of pump box 77. Various fluid connections may be made to permit adding CO₂ and/or air, sampling culture medium 1 and the biomass contained therein, and harvesting the biomass. One example of such connections is shown in FIG. 11 and described with reference to system 10 and another example of such connections is shown in FIG. 12 and described with reference to a system 10A that is like system 10 except for the modifications described below.

System 10 may utilize CO₂ and/or air supply 140 for adding gas to culture medium 1 and pump 34 for both culture medium 1 to vessel 52 and sampling or harvesting the biomass contained in vessel 52. As shown in FIGS. 7, 8, 10, and 11, for example, a line 100 including an in-line filter 101 may establish a first fluid communication between a first port of three-way valve 99 and CO₂ and/or air supply 140; a line 102 may establish a second fluid communication between a second port of three-way valve 99 and port 39; a line 103 may establish a third fluid communication between a third port of three-way valve 99 and a first port of a Y-connector 104 leading to port 38; a line 105 may establish a fourth fluid communication between a second port of Y-connector 104 leading to port 38 and a first port of pump 34; a line 106 may establish a fifth fluid communication between a second port of pump 34 and a first port of a Y-connector 107 leading to a line 108 that directs an output flow of culture medium 1 toward an outlet or reservoir 109; and a line 110 with an in-line filter 111 may establish a sixth fluid communication between a second port of Y-connector 107 and culture medium supply 150.

Aspects of the FIG. 11 connections for may be optimized to harvest, sample, and add culture medium 1 in a biosecure manner. As shown in FIG. 11, for example, line 108 may comprise a one-way check valve 112 and line 110 may comprise a one-way check valve 113, making it possible to switch system 10 between: a gas addition mode, in which the second port of three-way valve 99 is closed and CO₂ and/or air supply 140 is operable to add CO₂ and/or air to the bottom of vessel 52 via port 38 without introducing contaminants thereto (e.g., because of filter 101); a medium addition mode, in which pump 34 is operable with check valve 112 closed and check valve 113 open to add a volume of culture medium 1 to vessel 52 through port 38 without introducing contaminants thereto (e.g., because of filter 111); and a medium removal mode, in which pump 34 is operable with check valve 112 open and check valve 113 closed to harvest or sample culture medium 1 by pumping it out of vessel 52 through port 38, Y-connector 107, and line 108 toward reservoir 109 without introducing contaminants thereto (e.g., without opening vessel 52).

System 10A may utilize pump 34 for adding culture medium 1 to vessel 52 and CO₂ and/or air supply 140 for adding gas to culture medium 1 in vessel 52 and sampling or harvesting the biomass contained in vessel 52. As shown in FIG. 12, for example, line 100 including its in-line filter 101 may establish the aforementioned first fluid communication between the first port of three-way valve 99 and CO₂ and/or air supply 140; a line 102A may establish a second fluid communication between a second port of three-way valve 99 and a first port of a Y-connector 104A leading to port 39; a line 103A may establish a third fluid communication between a third port of three-way valve 99 and a first port of a Y-connector 107A leading to port 38; a line 105A may establish a fourth fluid communication between a second port of Y-connector 104A leading to port 39 and a first port of pump 34; a line 106A may establish a fifth fluid communication with a first port of a Y-connector 107A that directs an output flow of culture medium 1 into a culture medium reservoir 109; and a line 110A that establishes a sixth fluid communication between a second port of pump 34 and culture medium supply 150.

Aspects of the FIG. 12 connections also may be optimized to harvest, sample, and add to culture medium 1 in a biosecure manner. As shown in FIG. 12, for example, system 10A may be similarly switchable between: a gas addition mode, in which the second port of three-way valve 99 is closed and CO₂ and/or air supply 140 is operable to add CO₂ and/or air to the bottom of vessel 52 via port 38 without introducing contaminants thereto (e.g., because of filter 101); a medium addition mode, in which the second port of three-way valve 99 is closed and pump 36 is operable to add culture medium 1 to vessel 52 through lines 110, line 105A, and the second port of Y-connector 107A without introducing contaminants to vessel 52 (e.g., because of filter 111); and a medium removal mode, in which the third port of three-way valve 99 is closed, check valve 112 is open, and CO₂ and/or air supply 140 is operable to pressurize a head space of vessel 52 above culture medium 1, causing a flow of medium 1 and shredded biomass contained therein to exit vessel 52 through port 38, Y-connector 107A, and line 106A toward culture medium reservoir 109 without introducing contaminants thereto (e.g., without opening vessel 52).

As shown in FIGS. 11 and 12, for example, filter 101 and/or filter 111 may comprise an in-line UV-C LED sterilizer operable to sterilize the flow of air, CO₂, and/or new culture medium 1 (e.g., water) flowing into system 10. The in-line sterilizer may be installed using hose barbs and/or tri-clamp ends and autoclavable with the other lines described herein.

As shown in FIG. 2, for example, after the above-noted connections have been made, front wall 44 may be attached to floor plate 41 and front top plate 45 to prevent ambient light from reaching vessel 52. As shown in FIGS. 7 and 8, for example, aspects of light-tight enclosure 30 may be important to the efficient production of gametophytes. For example, an alternative approach may be to locate housing 31 in a room and obtain light spectrum control over the entire room, such as by putting the entire room under red light to expose the culture medium 1 inside of housing 31 to the red light. This alternative approach works fine until it becomes necessary to change culture medium 1 to “ph level” by exposing it to blue light, which would otherwise require disconnecting and moving housing 31 into another room and/or draining vessel 52 and moving culture medium 1 to another vessel 52 in another room, both of which may negatively affect growth rates.

Aspects of these exemplary assembly methods may be applicable to seaweed bioreactor apparatus 221, seaweed bioreactor apparatus 421, and any variations therefore with appropriate modifications for the structural differences noted above. Because some aspects of seaweed bioreactor apparatus 221, 421 are simpler than counterpart elements of seaweed bioreactor apparatus 21, for example, it is completed that some of the methods described above also may be simplified accordingly.

### Exemplary Methods of Operating Seaweed Bioreactor Apparatus and Systems

Aspects of operating seaweed systems (e.g., systems 10, 200), seaweed bioreactor systems (e.g., systems 210, 210*, 212, 212*, 213, 214), seaweed bioreactors (e.g., such as bioreactors 20, 220, 420), and seaweed bioreactor apparatus (e.g., such as bioreactor apparatus 21, 22, 23, 24, 221, 222, 223, 224, 421, 422, 423, 444) are now described with reference to a different seaweed methods including a seaweed method 1000, a seaweed method 1100, a seaweed method 1200, and a seaweed method 1300, each of which may be performable with microcontroller 40 and/or system controller 160 or life support systems 500, 501, 502, 501 and/or system controller 260.

As shown in FIG. 14, for example, seaweed method 1000 may comprise operating CO₂ and/or air supply 140, air supply control valve 142, CO₂ supply 143, CO₂ supply valve 144, and/or three-way valve 99 to continuously introduce a mixture of air and/or CO₂ into the bottom portion of vessel 52 through port 38. Method 1000 may therefore be utilized to circulate air throughout culture medium 1 on a regular basis, helping to optimize and sustain a seaweed biomass in culture medium 1 like gametophytes and/or sporophytes.

As shown in FIG. 15, for example, seaweed method 1100 may comprise: operating motor 33 to continuously rotate blade 67 in a mixing direction at a predetermined rate (e.g., such as approximately 50 to approximately 360 RPM) to stir culture medium 1 with mixing or scimitar-shaped edges 74 of blade 67 (a mixing step 1110); and intermediately rotating blade 67 in an opposite or shredding direction at a faster predetermined rate (e.g., such as approximately 2,000 to 4,000 RPM) to shred the seaweed cells contained in culture medium 1 with shredding or sickle-shaped edges 73 of blade 67 (a shredding step 1120). Step 1110 may swirl culture medium 1 and the biomass of gametophytes and/or sporophytes contained therein in the mixing direction and step 1120 may be utilized to increase the shredding capabilities of shredding or sickle-shaped edges 73 by rotating them in the shredding direction while the biomass is still swirling in the mixing direction.

As shown in FIG. 16, for example, seaweed method 1200 may comprise maintaining a desired metabolic rate of the seaweed biomass in culture medium 1 based on its demand for CO₂ by: monitoring a pH level of culture medium 1 with culture medium sensor 35 (a monitoring step 1210); and modifying CO₂ supply 143 and/or CO₂ supply valve 144 to maintain a desired pH level (a modifying step 1220). Method 1200 may comprise utilizing CO₂ consumption feedback to optimize production by determining an optimum harvest rate required to maximize CO₂ consumption. Different variables may be adjusted with modifying step 1220. Seaweed biomass is about 80% carbon, so the more CO₂ that system 10 needs, the more biomass it's producing, allowing any parameter of system 10 (e.g., temperature and/or nutrients) to be further adjusted based on the amount of CO₂ that must be introduced in step 1220 to maintain a desired pH level.

Seaweed method 1200 also may comprise automatically modifying the intensity and/or type of light output from light source 36 based on the CO₂ demand. For example, manipulation step 1220 of FIG. 16 may comprise selecting a maximum intensity and/or type of light output from light source 36 that a particular seaweed biomass in culture medium 1 can handle before it becomes photo-inhibited, which (e.g., like a sunburn) occurs when the light negatively affects the photoreceptors (e.g., chlorophyll A & B), such as by causing “stubby receptors” that take a few days to grow back. Because of culture medium sensor 35, seaweed method 1200 may therefore solve a recurring problem with manual control, in which photo-inhibition is unknowingly caused by a human operator, leading to a drop in the growth rate that could easily be resolved by reducing the light for a few days if the human operator understood why the drop occurred. In many instances, because culture medium sensor 35 is absent, the human operator cannot obtain this understanding and will either do nothing or (even worse) increase the intensity of the light, causing all or a substantial portion of the seaweed biomass in culture medium 1 to die.

As shown in FIG. 16, for example, manipulation step 1220 also may comprise: increasing an intensity of light output from source 36 by a known percentage (e.g., 2%) (an increasing step 1240); determining how the increase has affected CO₂ demand (a determining step 1250); and either: increasing the intensity of the light output from source 36 by another known percentage (e.g., another 2%) if the CO₂ demand is higher (a second increasing step 1260), indicating that the culture is happy; or reducing the intensity of the light by another known percentage (e.g., by 1%) if the CO₂ demand is lower (a decreasing step 1270), indicating that the culture is not happy and/or has been photo-inhibited. In this example, if the CO₂ demand does not increase after increasing step 1240, then second increasing step 1260 will not occur and reducing step 1270 may comprise: reducing the known percentage previously applied with increasing step 1240 (i.e., the above-referenced 2%) by a predetermined reset amount (e.g., by 50%); waiting for a predetermined amount of time (e.g., one hour); and starting the process over by removing the predetermined reset amount and/or returning to increasing step 1240.

Seaweed method 1200 may enhance the productivity of culture medium 1 by allowing system 10 and/or bioreactor 20 to iteratively determine a maximum possible light intensity that culture medium 1 can handle before photoinhibition. Seaweed method 1200 may be particularly useful when scaling up a culture, which often requires the operator to start with dim lights because there is only a small amount (e.g., 200 L) of dilute algae in the bottom, making it easy to give it too much light and kill the culture, in which even 10% light may be too much. A common manual practice is to “eyeball” the grow levels and manually increase the intensity of light output from light source 36 incrementally over time, such as by 5% or 10% each hour. These common manual practices become somewhat arbitrary as the biomass gets denser, leading to inefficiencies that may be avoided with manipulation step 1220 and steps 1240-1270 described herein.

As shown in FIG. 17, for example, seaweed method 1200 may comprise optimizing other parameters based on CO₂ demand, such as temperature and nutrients. For example, manipulation step 1220 may comprise performing a multivariable analysis and/or applying AI/machine learning to optimize light, harvest rate, temperature, nutrient, and the like based on outputs from culture medium sensor 35. System 10 and/or bioreactor 20 also may comprise additional sensing elements and manipulation step 1220 may be performed responsive to those elements. For example, seaweed bioreactor apparatus 21 may comprise a fluorescence probe operable to measure a fluorescence of the culture in vessel 52 and seaweed method 1200 may comprise automatically determining characteristics of a seaweed biomass in culture medium 1 with the fluorescence probe. As a further example, seaweed bioreactor apparatus 21 may comprise an optical sensing element, such as an optical camera mounted to the exterior of housing 31 adjacent a closed end of vessel 52; and seaweed method 1200 may comprise determining characteristics of the biomass in culture medium 1 by measuring a turbidity of medium 1 at the closed end of vessel 52.

As shown in FIG. 18, for example, seaweed method 1300 may comprise harvesting the seaweed biomass in culture medium 1, sampling the biomass, and/or adding additional volumes of gas and/or culture medium 1 to vessel 52 in a biosecure manner by preventing backflows that might otherwise contaminate the volume of culture medium 1 in vessel 52. Aspects of seaweed method 1300 may be performed with system 10 and 10A.

With respect to system 10, for example, seaweed method 1300 may comprise: adding CO₂ and/or air to the bottom of vessel 52 by closing the second port of three-way valve 99 and causing CO₂ and/or air supply 140 to deliver the CO₂ and/or air to vessel 52 via port 38; adding a volume of culture medium 1 to vessel 52 by closing check valve 112, opening check valve 113, and causing pump 34 to deliver the additional volume via port 38; and sampling or harvesting culture medium 1 by opening check valve 112, closing check valve 113, and causing pump 34 to deliver a flow of culture medium 1 and shredded biomass contained therein out of vessel 52 through port 38, Y-connector 107, and line 108 toward reservoir 109. With respect to system 10A, for example, seaweed method 1300 may comprise: adding CO₂ and/or air to the bottom of vessel 52 by closing the second port of three-way valve 99 and causing CO₂ and/or air supply 140 to deliver the CO₂ and/or air to vessel 52 via port 38; adding a volume of culture medium 1 to vessel 52 by causing pump 34 to deliver the additional volume to vessel 52 from culture medium supply 150 via port 39, line 105A, and line 110; and sampling or harvesting culture medium 1 by closing the third port of three-way valve 99, opening check valve 112, and causing CO₂ and/or air supply 140 to pressurize the head space of vessel 52 until a flow of culture medium 1 and shredded biomass contained exits vessel 52 through port 38, Y-connector 107A, and line 106A toward reservoir 109. Positively pressuring the headspace of vessel 52 in this manner also prevents contaminants from being sucked into vessel 52.

Aspects of these exemplary operating methods may be applicable to any seaweed systems, seaweed bioreactor systems, seaweed bioreactors, and/or seaweed bioreactor apparatus described herein with appropriate modifications for the structural differences noted above. Because some aspects of seaweed bioreactor apparatus 221, 421 are simpler than counterpart elements of seaweed bioreactor apparatus 21, for example, it is completed that some methods described above may be simplified accordingly.

While principles of the present disclosure are described herein with reference to illustrative aspects for particular applications, the disclosure is not limited thereto. Those having ordinary skill in the art and access to the teachings provided herein will recognize additional modifications, applications, aspects, and substitution of equivalents all fall in the scope of the aspects described herein. Accordingly, the present disclosure is not to be considered as limited by the foregoing description.

1. A method comprising:
sealing a seaweed biomass and a culture medium in a light-transmitting vessel located in a light-controlling enclosure; and
sampling or harvesting the seaweed biomass without unsealing the light-transmitting vessel by
causing, with a controller, a light source to output an artificial light into the light-controlling enclosure that illuminates the seaweed biomass,
outputting, with a sensor in communication with the controller, sensory data associated with the culture medium;
modifying, with the controller, at least one of the artificial light or the culture medium responsive to the sensory data;
intermittently mixing and cutting, with the controller, the seaweed biomass in the light-transmitting vessel, and
removing a portion of the seaweed biomass from the light-transmitting vessel and the light-tight enclosure.

2. The method of claim 1, wherein sealing the seaweed biomass comprises adding seaweed cells to the culture medium.

3. The method of claim 1, wherein sealing the seaweed biomass comprises adding seaweed cells to the culture medium without opening the light-controlling enclosure or unsealing the light-transmitting vessel.

4. The method of claim 2 or 3, wherein adding seaweed cells comprises adding one or more of diploids, seaweed haploid spores, female seaweed haploid adults, male seaweed haploid adults, seaweed eggs, seaweed sperm, seaweed sporophytes, seaweed zoospores, gametophytes, eggs, gametes, or sporophytes.

5. The method of claim 2 or 3, wherein adding seaweed cells comprises adding one or both of male gametophytes and female gametophytes.

6. The method of claim 1, wherein sealing the culture medium comprises maintaining a biosecure environment inside the light-transmitting vessel.

7. The method of claim 6, comprising adding an amount of water to the culture medium while maintaining the biosecure environment inside the light-transmitting vessel.

8. The method of claim 1, wherein sampling or harvesting the seaweed biomass comprises removing the portion of the seaweed biomass without introducing unfiltered ambient air to the light-transmitting vessel.

9. The method of claim 1, comprising only opening the light-controlling enclosure after a period spanning months or years.

10. The method of claim 9, comprising only unsealing the light-transmitting vessel after a period spanning months or years.

11. The method of claim 1, wherein causing the light source to output the artificial light comprises causing a LED to output the artificial light.

12. The method of claim 11, wherein causing the LED output the artificial light comprises causing a spectrum selectable LED to output the artificial light.

13. The method of claim 12, comprising:
causing, with the controllers, the spectrum selectable LED to output a first hue of the artificial light for a first period; and
causing, with the controllers, the spectrum selectable LED to output a second hue of the artificial light for a second period [[,]].

14. The method of claim 13, wherein outputting the second hue of the artificial light for the second period comprises changing the seaweed biomass to sexual.

15. The method of claim 1, wherein the light-controlling enclosure comprises a light-filtering window and the method comprises admitting a filtered portion of ambient light into the light-controlling enclosure through the light-filtering window.

16. The method of claim 15, wherein admitting the filtered portion of ambient light for the second period does not comprise changing the seaweed biomass to sexual.

17. The method of claim 1, wherein modifying the culture medium comprises modifying, with the controller, a flow of CO₂ to culture medium.

18. The method of claim 1, wherein outputting the sensory data comprises outputting a pH level of the culture medium.

19. The method of claim 18, comprising adjusting, with the controller, the at least one of the artificial light or the culture medium responsive to pH level of the culture medium.

20. The method of claim 19, comprising:
monitoring, with the controller, the pH level of the culture medium; and
modifying, with the controller, the culture medium by modifying a flow of CO₂ to the culture medium responsive to the pH level of the culture medium until it approaches a target pH level of the culture medium.

21. The method of claim 20, comprising:
determining, with the controller, a CO₂ demand of the seaweed biomass responsive to the pH level of the culture medium; and
modifying, with the controller, the culture medium responsive to the CO₂ demand.

22. The method of claim 21, comprising:
causing, with the controller, the light source to increase an intensity of the artificial light by a first percentage;
estimating, with the controller, a CO₂ demand of the seaweed biomass responsive to the increased intensity; and
modifying, with the controller, the flow of CO₂ responsive to the CO₂ demand.

23. The method of claim 22, comprising causing, with the controller, the light source to:
increase the intensity of the artificial light by a second percentage if the CO₂ demand increases responsive to the increased intensity; or
decrease the intensity of the artificial light by a third percentage if the CO₂ demand decreases responsive to the increased intensity.

24. The method of claim 23, causing, with the controller, the light source to decrease the intensity comprises:
decreasing the intensity of the artificial light by a predetermined reset amount;
waiting for a predetermined period; and
repeating the steps of:
monitoring, with the controller, the pH level of the culture medium; and
modifying, with the controller, the culture medium by modifying the flow of CO₂ to the culture medium responsive to the pH level of the culture medium until it approaches a target pH level of the culture medium.

25. The method of claim 1, wherein outputting the sensory data comprises outputting a temperature of the culture medium.

26. The method of claim 25, comprising adjusting, with the controller, the at least one of the artificial light or the culture medium responsive to the temperature of the culture medium.

27. The method of claim 26, comprising modifying the culture medium by exposing the light-transmitting vessel to a temperature regulating fluid in the light-controlling enclosure responsive to the temperature of the culture medium.

28. The method of claim 27, wherein exposing the light-transmitting vessel to the temperature regulating fluid comprises circulating the temperature regulating fluid around the light-transmitting vessel.

29. The method of claim 28, wherein exposing the light-transmitting vessel to the temperature regulating fluid comprises causing, with the controller, a pump to circulate an amount of water around the light-transmitting vessel.

30. The method of claim 28, wherein exposing the light-transmitting vessel to the temperature regulating fluid comprises causing, with the controller, a temperature regulation control unit to output a flow of conditioned air toward the light-transmitting vessel responsive to the temperature of the culture medium.

31. The method of claim 1, wherein intermittently mixing and cutting the seaweed biomass comprises cutting the seaweed mass with a cutting element sealed in the light-transmitting vessel.

32. The method of claim 1, comprising a blade sealed in the light-transmitting vessel, wherein intermittently mixing and cutting the seaweed biomass comprises causing, with the controller, a motor to cut the seaweed biomass by rotating the blade in a first direction relative to light-transmitting vessel.

33. The method of claim 32, wherein intermittently mixing and cutting the seaweed biomass comprises causing, with the controller, the motor to mix the seaweed biomass by rotating the blade in a second direction opposite the first direction.

34. The method of claim 33, comprising causing, with the controller, the motor to:
continuously rotate the blade in the second direction at a first RPM for a period; and
intermittently rotate the blade in the first direction at a second RPM during the period.

35. (canceled)

36. The method of claim 34, wherein the second RPM is greater than the first RPM.

37. The method of claim 32, wherein intermittently mixing and cutting the seaweed biomass comprises causing, with the controller, an aerator to mix the seaweed biomass by aerating the culture medium in the light-transmitting vessel.

38. The method of claim 1, wherein removing the portion of the seaweed biomass comprises pressurizing a head space of the light-transmitting vessel.

39. The method of claim 38, wherein pressurizing the head space comprises directing, with the controller, a flow of CO₂ to into the head space.

40. The method of claim 1, wherein outputting the sensory data comprises:
causing, with the controller, an optical sensor to determine an opacity of the culture medium; and
adjusting, with the controller, the at least one of the artificial light or the culture medium responsive to the opacity of the culture medium.

41. The method of claim 37, wherein causing the aerator to mix the seaweed biomass comprises passing a flow of CO₂ and/or air through a bottom of the light-transmitting vessel without introducing contaminents to the culture medium.
